SEQUENCE LISTING (14 sequences)

SEQ ID NO: 1 - DNA, 333 bp, Homo sapiens
gatatcttgctcacccaaactccagcttctttggctgtgtctctagggcagagggccaccatctcctgcaaggccagccaaagtgttgattatgatggtgatagttatttgaactggtatcaacagattccaggacagccacccaaactcctcatctatgatgcatccaatctagtttctggaatcccacccaggtttagtggcagtgggtctgggacagacttcaccctcaacatccatcctgtggagaaggtggatgctgcaacctatcactgtcagcaaagtactgaggacccgtggacgttcggtggaggcaccaagctggaaatcaaa

SEQ ID NO: 2 - protein, 111 aa, Homo sapiens
DILLTQTPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 3 - DNA, 321 bp, Artificial sequence
  Synthetic
gatattcaaatgacccagagtccaagtagtcttagtgccacactgggcggtaaagtgacgatcacttgtaaagccagccaggacatcaacaaatacatagcttggtatcagcataaaccgggaaagggaccgagactgctgatccattatacttccacgctccagcccggtaacccaagccgattttctgggagcggaagtggtagggattatagcttttccattagcaacttggaagccgaagacattgctatatactactgtctgcaatatgacaacttgcagcgcacttttggtgggggaaccaaagtggaaattaag

SEQ ID NO: 4 - protein, 107 aa, Artificial sequence
  Synthetic
DIQMTQSPSSLSATLGGKVTITCKASQDINKYIAWYQHKPGKGPRLLLHYTSTLQPGNPSRFSGSGSGRDYSFSISNLEAEDIAIYYCLQYDNLQRTFGGGTKVEIK

SEQ ID NO: 5 - DNA, 370 bp, Homo sapiens
caagtgcaactccaacagtcaggggctgagctggtgaggcctggctcctcagtgaagatttcctgcaaggcttctggctatgcattcagtagctactggatgaactgggtgaagcagaggcctggacagggtcttgagtggattggacagatttggcctggagatggtgatactaactacaggaaagttcaagggtaaagccactctgactgcagacgaatcctccagcacagcctacatgcaactcagcagcctagcatctgaggactctgcggtctatttctgtgcaagacgggagactacgacggtaggccgttattactatgctatggactactggggtcaaggaacctcagtcaccgtctcgagc

SEQ ID NO: 6 - protein, 124 aa, Homo sapiens
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVSS

SEQ ID NO: 7 - DNA, 339 bp, Artificial sequence
  Synthetic
cagatccaactcgtgcagtctggacccgagttgaagaaacccggtgagactgtaaagatttcttgcaaagcctccggctacacatttaccgattattccatgtactgggtgaagcaggcacctggaaaagggttgaaaaggatgggttggataaacacagaaaccggcgaaccaacctatgccgacgacttcaagggtcgattcgcgctttccctggatacttccgcctccaccgcataccttcacatttccaacctcaagaacgaagacacagctacatacttttgcgcccgaggacttgatagctggggtcagggtactagtgttaccgtctcgagc

SEQ ID NO: 8 - protein, 113 aa, Artificial sequence
  Synthetic
QIQLVQSGPELKKPGETVKISCKASGYTFTDYSMYWVKQAPGKGLKRMGWINTETGEPTYADDFKGRFALSLDTSASTAYLHISNLKNEDTATYFCARGLDSWGQGTSVTVSS

SEQ ID NO: 9 - DNA, 321 bp, Artificial sequence
  Synthetic
cgggctgatgcggcgccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacagaaatgagtgt

SEQ ID NO: 10 - protein, 106 aa, Artificial sequence
  Synthetic
RADAAPTVSIFPPSSEQLTSGGASWCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - DNA, 312 bp, Artificial sequence
  Synthetic
gccaaaacgacacccccatctgtctatccactggcccctggatctgctgcccaaactaactccatggtgaccctgggatgcctggtcaagggctatttccctgagccagtgacagtgacctggaactctgggtccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgcggatcc

SEQ ID NO: 12 - protein, 104 aa, Artificial sequence
  Synthetic
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGS

SEQ ID NO: 13 - DNA, 24 bp, Artificial sequence
  Synthetic
ggcggttccagcggcagcggctgc

SEQ ID NO: 14 - DNA, 8 bp, Artificial sequence
  Synthetic
ggssgsgc

(19) United States
(12) Patent Application Publication
(10) Pub. No.: US 2024/0033373 A1
(43) Pub. Date: Feb. 1, 2024

(54) TARGETED, INTRACELLULAR DELIVERY OF THERAPEUTIC PEPTIDES USING SUPRAMOLECULAR NANOMATERIALS

(71) Applicant: The University of Chicago, Chicago, IL (US)

(72) Inventors: Matthew Tirrell, Chicago, IL (US); James L. Labelle, Chicago, IL (US); Mathew Schnorenberg, Chicago, IL (US); Jeffrey A. Hubbell, Chicago, IL (US); Elyse A. Watkins, Chicago, IL (US)

(21) Appl. No.: 18/043,286

(22) PCT Filed: Aug. 31, 2021
(86) PCT No.: PCT/US2021/048492

§ 371 Date: Feb. 27, 2023

Related U.S. Application Data
(60) Provisional application No. 63/072,286, filed on Aug. 31, 2020.

(51) Int. Cl. A61K 47/69; A61K 47/68; A61K 45/06; A61P 35/00

(52) U.S. Cl. CPC A61K 47/6915; A61K 47/6849; A61K 45/06; A61P 35/00

(57) ABSTRACT

Provided herein are nanoparticles to encapsulate therapeutic agents and methods of use thereof for intracellular delivery and disease treatment. In particular, the present disclosure provides polymersomes for use in the delivery of therapeutic agents, e.g. stapled peptides, to diseased (e.g. cancer) cells.

### CROSS-REFERENCE TO RELATED APPLICATION

The present application claims priority to U.S. Provisional patent application Ser. No. 63/072,286, filed Aug. 31, 2020, which is hereby incorporated by reference in its entirety.

### STATEMENT REGARDING FEDERALLY SPONSORED RESEARCH OR DEVELOPMENT

This invention was made with government support under CA221250 awarded by the National Institutes of Health. The government has certain rights in the invention.

### SEQUENCE LISTING

The text of the computer readable sequence listing filed herewith, titled “38732-601_SEQUENCE_LISTING_ST25”, created Apr. 16, 2021, having a file size of 10,336 bytes, is hereby incorporated by reference in its entirety.

### FIELD

The present disclosure relates to nanoparticles for encapsulation of therapeutic agents and methods of use thereof for intracellular delivery and disease treatment. In particular, the present disclosure provides polymersomes for use in the delivery of therapeutic agents, e.g. stapled peptides, to diseased (e.g. cancer) cells.

### BACKGROUND

Despite their known biomedical importance, intracellular protein-protein interactions (PPIs) have long been considered “undruggable” therapeutic targets using traditional, “drug-like” small molecules (Refs. 1-2; incorporated by reference in their entireties) because most PPI interfaces are significantly larger than the surface areas bound by small molecules drugs. Hydrocarbon-stapled peptides are promising tools for disrupting PPIs. Hydrocarbon-stapled peptides mimic a PPI interface through stabilization of a natural α-helical secondary structure while imparting it with drug-like properties including enhanced binding specificity, affinity, protease resistance, and in some cases cellular uptake (Refs. 8-14; incorporated by reference in their entireties).

However, significant obstacles remain for the clinical translatability of stapled peptides, including achieving cellular uptake at therapeutically-relevant concentrations into the diseased cells of interest. Highly-optimized, cell-penetrating stapled peptides still typically require 100-10,000 times higher concentrations for efficacy in assays in which the cell membrane is intact (e.g. in vitro cellular assays) than in assays in which the cell membrane is absent or permeabilized (e.g. ex vitro protein binding assays, mitochondrial depolarization assays) (Refs. 21-23; incorporated by reference in their entireties). Moreover, research-grade stapled peptides are often sequestered and completely inhibited by serum proteins (Refs. 22, 24-26; incorporated by reference in their entireties), and the same modifications that make them cell permeable make them insufficiently water soluble for intravenous injection.

### SUMMARY

Experiments conducted during development of embodiments herein demonstrate that polymersomes modified with targeting moieties can be used for the delivery of therapeutic agents, e.g. stapled peptides, to cells which were previously unable to be delivered effectively. The polymersomes described herein allow specific uptake into the cells or tissues of interest and decrease the therapeutic effective amount needed.

In some embodiments, the polymersomes comprise a plurality of amphiphilic disulfide block co-polymers; a targeting moiety conjugated on an exterior surface of the polymersome to a portion of the plurality of amphiphilic disulfide block co-polymers; and an encapsulated cargo molecule. The polymersome may be capable of releasing the encapsulated cargo molecule inside an endosome.

In some embodiments, the targeting moiety comprises an antibody or fragment thereof. In some embodiments, the targeting moiety binds to a cell surface protein (e.g. CD19). In some embodiments, the targeting moiety further comprises a cysteine linker. The targeting moiety may be conjugated to less than 1% of the plurality of amphiphilic disulfide block co-polymers. In some embodiments, the moiety may be conjugated to 0.01-1% of the plurality of amphiphilic disulfide block co-polymers

The encapsulated cargo molecule may comprise a therapeutic agent, a marker, or a combination thereof. In some embodiments, the encapsulated cargo molecule comprises a therapeutic agent. In some embodiments, the encapsulated cargo molecule comprises a stapled peptide. The stapled peptide may be a hydrophobic stapled peptide, a hydrocarbon stapled peptide, and/or may comprise polar and/or charged functional groups.

In some embodiments, the amphiphilic disulfide block co-polymers comprise poly(ethylene glycol) (PEG) and poly(propylene sulfide) (PPS).

The disclosure also provides compositions comprising a polymersome described herein and methods of using the polymersomes or compositions thereof for treating a disease or disorder (e.g. cancer) or targeting a therapeutic agent to a desired location within a subject.

### BRIEF DESCRIPTION OF THE DRAWINGS

FIG. 1 is a schematic of CD19-targeted polymersomes deliver SAH-MS1-18 into the cytoplasm of human diffuse large B-cell lymphoma (DLBCL) cells to reactivate cell death and synergize with p53-reactivation. (a) Cancer cells rely on PPIs for inhibition of apoptosis (e.g. MCL-1 sequesters pro-apoptotic proteins). (b) Therapeutic stapled peptides (e.g. SAH-MS1-18) can potently and specifically block a disease-driving PPI. (c) Cellular uptake is a major obstacle to the clinical translation of therapeutic stapled peptides. (d) Stapled peptides are stably encapsulated in PEG-SS—PPS polymersomes. (e) Recombinant αCD19 Fabs are functionalized with a site-specific click chemistry handle. (f) The polymersomes are decorated with αCD19 Fabs and the targeted polymersomes (αCD19-PSOMs) purified. (g) αCD19-PSOMs bind CD19 on DLBCL cells and initiate endocytosis. (h) In the relatively reducing endosome, the disulfide of PEG-SSPPS is reduced. (i) Polymersomes are disrupted and release their cargo. (j) The hydrophobic PPS block facilitates endosomal escape. (k) SAH-MS1-18 binds MCL-1 in the cytoplasm to release pro-apoptotic proteins and (1) reactivate apoptosis if the cell is sufficiently primed to die. (m) Systemic treatment with the p53-reactivating stapled peptide ATSP-7041 (n) inhibits p53's inhibitory binding partners. (o) In cancer cells, phosphorylated/activated p53 translocates to the nucleus to upregulate transcription of pro-apoptotic proteins (e.g. PUMA, BAX) and downregulate transcription of anti-apoptotic proteins (e.g. BCL-2). (p) p53 transcriptional changes sensitize DLBCL to cell death by MCL-1 inhibition.

FIGS. 2A-F show PEG-SS—PPS polymersome assembly, characterization, and stability. FIG. 2A is a schematic of the synthesis of the PPS polymer block by living, anionic, ring-opening polymerization (i-iii) followed by disulfide reduction (iv) and capping with a pyridyl disulfide functional group (v) to generate PPS-PDS (compound 1). PPS-PDS was then reacted with thiolated PEG polymers (vi) to generate PEG-SS—PPS block copolymers with methoxy (OMe; compound 2) or azide (N3; compound 3) end groups. PEG-SS—PPS block copolymers were then assembled into polymersomes. FIG. 2B is graphs of DLS measurements of empty polymersomes formed by a thin film method (“Thin Film”) or by flash nanoprecipitation (“FNP”), followed by extrusion through a 100 nm pore-size membrane (“Extrusion”) and desalting into PBS (“SEC”). DLS measurements were repeated until the residuals of the average correlation function fit were negligible (10-120 times). Plotted are the intensity-scaled size distribution from the Regularization fit method. Dh and PDI are given for the SEC-purified samples. FIG. 2C is cryo-EM images confirming the polymersomes are uniform, hollow spheres with diameters and bilayer thicknesses that correspond to DLS and SAXS measurements. Scale bars are 100 nm. FIG. 2D is a graph of SAXS data fit to hollow sphere structures at an ensemble level for both thin-film- and ash-nanoprecipitation-formed polymersomes. Intensity (a.u.) values are shown vertically shifted to prevent overlap of the plots. Polymersomes encapsulating a self-quenching calcein solution were diluted into various solutions, and fluorescence dequenching due to polymersome disruption was monitored for 1 hour at 37° C. (FIG. 2E). Data plotted are individual quadruplicates, each background subtracted against samples in which an equivalent volume of PBS-blank was added instead of polymersomes. FIG. 2F are chromatographs of aqueous SEC HPLC of free SAH-MS1-18 peptide (blue, dashed) compared to a polymersome solution encapsulating an equimolar amount of SAH-MS1-18 stored for one month at 4° C. in PBS (red, solid).

FIGS. 3A-3C show characterization of compound 1 (PPS-PDS). FIG. 3A is gel permeation chromatography (GPC) refractive index (RI) traces of PPS polymerization kinetics over time. From right to left, aliquots were taken at 15, 45, and 90 min, quenched with acetic anhydride, precipitated, and analyzed by GPC. Additional monomer was injected immediately after 45 min, and both the numeric thiol peak (PPS—SH) and dimeric disulfide peak (PPS—SS—PPS) continued growing, suggesting that disulfide exchange in the reaction is fast enough that disulfides did not significantly inhibit the polymerization. FIG. 3B is SEC RI traces of a completed PPS polymerization reaction with and without using tributylphosphine (TBP) to reduce disulfide chains (PPS—SS—PPS) to free thiol chains (PPS—SH). The dispersity of reduced PPS—SH was 1.17. FIG. 3C is the 1H NMR spectra of PPS-PDS (compound 1) in CDCl₃. The density of pure PPS-PDS was measured to be 1.169 g/mL.

FIGS. 4A and 4B shows characterization of compound 2 (mPEG-SS—PPS). FIG. 4A is the GPC RI trace of mPEG-SS—PPS (compound 2). Dispersity=1.08, with no contamination from either polymer block. FIG. 4B is the 1H NMR spectra of mPEG-SS—PPS (compound 2) in CDCl₃. One of the benzylic protons overlapped with the CDCl₃ peak and could not be integrated.

FIGS. 5A and 5B show the characterization of compound 3 (N3-PEG-SS—PPS). FIG. 5A is the GPC RI trace of N3-PEG-SS—PPS (compound 3). Dispersity=1.06, with no contamination from either polymer block. The commercially-available N3-PEG-SH had a large percentage of disulfide-dimerized chains (N3-PEG-SS-PEG-N3). The disulfide chains were considered to be inert bystanders in the reaction and would be removed during later purification steps (namely MeOH extraction). FIG. 5B is the 1H NMR spectra of N3-PEG-SS—PPS (compound 3) in CDCl₃. One of the benzylic protons overlapped with the CDCl₃ peak and could not be integrated.

FIGS. 6A-6C show the LCMS analysis of therapeutic peptides. After purification, the purity and identity of all peptides was confirmed by LCMS analysis. Representative chromatograms of UV absorbance at 220 nm are shown for SAH-MS1-18 (FIG. 6A), ATSP-7041 (FIG. 6B), and BIM-SAHB (FIG. 6C). Peptides diluted from DMSO stock solutions show a DMSO solvent injection absorbance. Stapled peptides with (i, i+7) staples, such as ATSP-7041, often have two isomers of the staple, as previously described by others, which elute as separate chromatographic peaks after stapling but have identical mass spectra. Ac=acetylated N-terminus. Am=amide C-terminus. B=norleucine. X=S5. Z=R8. Cba=β-cyclobutyl-L-alanine.

FIGS. 7A-7D show flash nanoprecipitation using a 3D-printed confined impingement jets with dilution (CIJ-D) device. FIGS. 7A and 7B are cut-away views of the 3D-printed CAD design using the same dimensions published previously (Ref. 50; incorporated by reference in its entirety). FIG. 3C is an image of syringes attached to the CIJ-D device inlets via threaded Luer-lock adapters, and an outlet tube placed into a PBS dilution reservoir. After rapid mixing, an air cushion in the syringes cleared the device and mixed the dilution reservoir with air bubbles. FIG. 7D is an image of the resulting opaque polymersome solution which resulted even when the polymersomes are smaller than the wavelength of light due to their very high concentration.

FIGS. 8A-8C show encapsulation of some drugs affects polymersome assembly. Polymersomes made from PEG-SS—PPS block copolymers typically had a primary population with Dh of 120-130 nm, and a 100 nm extrusion step broke up any larger aggregates to that same size. SAH-MS1-18 encapsulation at peptide:polymer mass ratios of 1:4 repeatedly produced polymersomes that were slightly smaller than typical polymersomes (FIG. 8A).

Two representative encapsulations are shown. S63845 encapsulation at high mass ratios produced micelles (as confirmed by cryo-EM), while a lower mass loading encapsulation via FNP produced typical polymersomes (FIG. 8B). ATSP-7041 at high mass loading ratios produced a mixed population of (presumably) micelles and polymersomes, with mostly micelles (FIG. 8C). Decreasing the mass loading ratio allowed the formation of normal polymersomes. All DLS data are intensity-scaled size distributions with Dh calculated from the Regularization fit.

FIG. 9 is DNA coding sequences of engineered Fabs and their protein translations. Fabs were designed with variable domains (V_{κ} and V_{H}) for binding to either human CD19 (αCD19) or an irrelevant xenoantigen (αOspA). All Fabs shared the same constant domains (C_{κ} and C_{H}). For αCD19-cys and αOspA-cys, the cysteine linker sequence was added to the C-terminal end of the CH domain.

FIGS. 10A-10C show expression and binding validation of Fabs. FIG. 10A is a schematic Fabs designed using previously published sequences as in FIG. 9 from antibodies that bind either human CD19 (αCD19) or an irrelevant xenoantigen (αOspA). To enable site-specific conjugation to polymersomes, a flexible cysteine linker was encoded at the C-terminus of the heavy chain of each Fab to generate αCD19-cys and αOspA-cys. FIG. 10B is a Coomassie staining on an SDS-PAGE gel of purified Fabs. Each Fab appears pure at the expected molecular weights, and addition of DTT in the loading buffer reduced the interchain disulfide to generate polypeptides (heavy chain and light chain) that overlap at their expected molecular weights. FIG. 10C is flow cytometry measurement of Fab binding to a CD19+ DLBCL cell line, SU-DHL-5. Cells were stained with the indicated Fab, then with an AF647-labeled αFab secondary antibody. αCD19 Fabs bound CD19+DLBCL with or without the cysteine linker, and the control (αOspA) Fabs did not.

FIGS. 11A-11E show Fab functionalization for attachment to polymersomes. Disulfide-capped Fabs were (i) reduced with TCEP (90 minutes at 37° C.), then (ii) immediately, without workup, reacted with a 100-fold excess of the heterobifunctional linker, Sulfo DBCO-PEG4-Maleimide, for 1 hour at room temperature (FIG. 11A). Excess linker was then removed by extensive diafiltration (10 kDa MWCO Amicon). A range of TCEP stoichiometries was used to determine the optimal amount of TCEP for reducing the cysteine linker without disrupting internal disulfides (FIGS. 11B and 11C). The DBCO:Fab ratio was determined by UV-vis absorbance (FIG. 11B), and the percent of intact Fab was determined by quantification of a Coomassie-stained SDS-PAGE gel (FIG. 11C). The y-values were normalized to the ratio of Fab in its intact, numeric form before the reaction (in this case, 80%), as measured by SDS-PAGE gel quantification. From these data, the reliable range (0.5-1 equivalents) of TCEP to reduce only the terminal cysteine linker and functionalize it with DBCO was determined. Using this optimized DBCO-functionalization protocol, αCD19-DBCO and αOspADBCO with DBCO:Fab ratios reliably ˜1. To attach Fabs to the polymersomes, polymersomes were assembled with 5% N3-PEG-SS—PPS and 95% mPEG-SS—PPS (FIGS. 11D and 11E). Fab-DBCO was added to react overnight, and then any non-conjugated Fab was removed by size (SEC or TFF diafiltration). In this example, enough DBCO was added to theoretically functionalize 0.10% of the polymer chains on the external polymersome surface (or 0.05% of the total polymer chains in the sample). After purification, the CBQCA protein quantification assay was used to detect Fab retained in the final samples, accounting for background signal contributions from blank, peptide-only, and empty polymersome samples (FIG. 11D). The polymer and peptide concentration of every sample was known from GPC and LCMS measurements, respectively, to calculate their relative background contributions. From this, the fluorescence contribution from Fab was calculated (purple bars), and the unknown Fab concentrations were calculated by comparing to the Fab-only control sample. To confirm the Fab remaining in the samples (as detected in (FIG. 11D) was attached to the polymersomes and not just contaminating, non-conjugated Fab, a Coomassie-stained SDS-PAGE gel was used to confirm the disappearance of the Fab-DBCO band (FIG. 11E). Importantly, this band disappearance was due to covalent, rather than non-covalent, Fab:polymer interaction, because spiking more Fab (the same amount as the Fab-only lane) into the polymersome samples restored the Fab band. Gel samples were loaded such that, assuming 100% Fab conjugation, the Fab bands would be identical. All gel samples were prepared in the presence of sodium azide (to quench DBCO:azide reactions) and NEM (to quench thiols and disulfide shuffling).

FIGS. 12A-12D show CD19 targeting enhances polymersome delivery into DLBCL cells. A self-quenching calcein solution was encapsulated in PEG-SS—PPS polymersomes with 5% N3 functionalization. Aliquots of this stock solution were then functionalized with either αCD19 or irrelevant (αOspA) Fabs at various Fab:polymer densities (+++, ++, +). DLBCL cell lines were treated as indicated and analyzed by flow cytometry and imaging cytometry. Treatment concentrations were normalized by calcein absorbance after Triton X-100 disruption and calcein dequenching. Uptake of fluorescent polymersomes into four DLBCL cell lines was measured by flow cytometry to evaluate time-, concentration-, Fab-, and Fab-density-dependence (FIG. 12A). αCD19 Fab functionalization greatly improved cellular uptake, and lower Fab densities caused more uptake. In FIG. 12B the same samples from FIG. 12A were subsequently analyzed by ImageStream imaging cytometry for single-cell fluorescence images. Representative images are shown with the following channels: brightfield, calcein (green), anti-Fab extracellular staining (magenta), and an overlay. CD19-specific polymer uptake correlates with CD19 expression (FIG. 12C). Cells were either stained with fluorescent αCD19 IgG or treated with αCD19-PSOMcalcein or αOspA-PSOM_{calcein} for 24 hours. An unstained, untreated sample of SU-DHL-5 is shown for comparison. Polymersome-uptake after 24 hours (FIG. 12D) was dose-dependent for both specific uptake (αCD19) and non-specific uptake (αOspA). The total polymer concentration in the treatment is indicated in μg/mL.

FIGS. 13A-13C show calcein uptake heatmaps. In FIG. 13A—top, the data measuring uptake of calcein-loaded polymersomes in OCI-Ly3 as shown in FIG. 12A. (In FIG. 13A—bottom) was re-scaled to visualize CD19-specific uptake in OCI-Ly3. Data as shown in FIG. 12A measuring uptake of calcein-loaded polymersomes in SU-DHL-5 (FIG. 13B). A similar experiment to FIG. 13B was conducted without the final purification step in which excess Fab was removed from the treatments (FIG. 13C). Excess Fab blocked nearly all antigen-specific uptake. Of note, the highest concentration treatments in (FIG. 13C) were 5-times higher than in (FIG. 13B) in terms of calcein concentration.

FIGS. 14A and 14B show polymersome delivery enhances the therapeutic potency of SAH-MS1-18 in DLBCL. When SAH-MS1-18 was delivered into SU-DHL-5 DLBCL cells using polymersomes, its potency was amplified by orders of magnitude (FIG. 14A). When the cells were treated with the same materials but formulated with free peptide on the outside of empty polymersomes, the therapeutic effect was completely eliminated. Across four different DLBCL cell lines, polymersome delivery enhances the therapeutic efficacy of SAH-MS1-18 (FIG. 14B). Plotted points are the means of duplicates+/−S.E.M. fitted to a normalized non-linear regression with variable slope.

FIG. 15 shows αCD19-PSOM delivery enhances the potency of BCL-2 family pan-activator, BIM-SAHB. BIM-SAHB was delivered to DLBCL cells either as free peptide, in CD19-targeted polymersomes, or in irrelevantly-targeted polymersomes, and viability was measured by CellTiter-Glo 2.0. Plotted points are the mean of duplicates+/−S.E.M. and fitted by normalized non-linear regression with variable slope (GraphPad Prism).

FIG. 16 is graphs of cell death sensitivities of DLBCL cell lines to ATSP-7041. Four DLBCL cell lines with WTp53 and two with mutant p53 (OCI-Ly1 and OCI-Ly8) were treated with ATSP-7041 at a range of doses for 24 or 72 hours when viability was measured using CellTiter Glo 2.0 relative to an untreated control. DMSO controls were included with a volume of DMSO equal to the highest peptide treatments. Data plotted are the mean of duplicates+/−S.E.M. fitted to a normalized non-linear regression with variable slope (GraphPad Prism 8).

FIGS. 17A-17E show p53-reactivation with ATSP-7041 primes DLBCL for apoptosis, particularly through MCL-1 inhibition. DLBCL cell lines were treated for 24 hours with either ATSP-7041 or vehicle control (DMSO) to assess the effects of p53-reactivation on the BCL-2 family of proteins. The relative mRNA expression levels of DLBCL cell lines with and without p53-reactivation were quantified for the BCL-2 family members and for p53's classic transcriptional target, CDKN1A/p21 (FIG. 17A). Plotted values are the mean of biological triplicates (each in technical triplicate)+/−S.E.M. Bands of a western blot of PUMA protein in DLBCL cell lines with or without p53-reactivation were quantified in ImageJ, normalized to actin, and quantified as the ratio of PUMA in the ATSP-7041 treatment to the vehicle control (FIG. 17B). FIG. 17C are graphs of apoptotic priming with or without p53-reactivation. After pre-treatment with ATSP-7041, mitochondrial depolarization was measured in response to varying doses of BIM BH3 peptide. A t-test was used to compare each pair of points. *p<0.005. FIG. 17D is a graph of the sensitivities to a BCL-2 inhibitor (ABT-199), BCL-XL inhibitor (A-1331852), and MCL-1 inhibitor (S63845) were measured with (+) or without (−) prior p53-reactivation by ATSP-7041. Dilution curves were made in duplicate, normalized to an untreated control receiving the same pre-treatment, and analyzed by non-linear regression to calculate the IC50+/−S.E. Individual dose curves are presented in FIG. 18. FIG. 17E are graphs of the cell death sensitivities to SAH-MS1-18 delivered in polymersomes with or without p53-reactivation. Plotted values are the mean of duplicates+/−S.E.M., normalized to untreated control and fitted using non-linear regression.

FIGS. 18A and 18B are graphs of DLBCL sensitivities to BH3-mimetics with and without p53 priming. Each cell line was treated for 24 hours with either ATSP-7041 or vehicle control (DMSO), washed, then treated for 24 hours with the indicated BH3 mimetic. Plotted points are means of duplicates+/−S.E.M., normalized to an untreated control that received the same pre-treatment, and fitted using nonlinear regression.

FIG. 19 shows polymersome delivery to DLBCL cells in vivo: pilot experiment. αCD19-PSOMcalcein delivers calcein to OCI-Ly8 DLBCL cells in both disseminated (bone marrow) and orthotopic (subcutaneous tumor) xenograft models in NSG mice. Mice were engrafted with OCI-Ly8 on day 0, treated once with αCD19-PSOM_{calcein} on day 6, and the DLBCL cells analyzed by flow cytometry on day 7. OCI-Ly8 cells were gated by size and CD19+CD20+ staining. N=2 mice per group. Plotted are the mean and range of the MFI.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing”, and “introducing,” are used interchangeably herein and refer to the placement of therapeutic agents into a subject by a method or route which results in at least partial localization a desired site. The therapeutic agents can be administered by any appropriate route which results in delivery to a desired location in the subject.

“Antibody” and “antibodies,” as used herein, refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al. (2014) Trends in Biotechnology 21:484-490), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2).

As used herein, the term “chemotherapeutic” or “anti-cancer drug” includes any drug used in cancer treatment or any radiation sensitizing agent. Chemotherapeutics may include alkylating agents (including, but not limited to, cyclophosphamide, mechlorethamine, chlorambucil, melphalan, dacarbazine, nitrosoureas, and temozolomide), anthracyclines (including, but not limited to, daunorubicin, doxorubicin, epirubicin, idarubicin, mitoxantrone, and valrubicin), cytoskeletal disrupters or taxanes (including, but not limited to, paclitaxel, docetaxel, abraxane, and taxotere), epothilones, histone deacetylase inhibitors (including, but not limited to, vorinostat and romidepsin), topoisomerase inhibitors (including, but not limited to, irinotecan, topotecan, etoposide, teniposide, and tafluposide), kinase inhibitors (including, but not limited to, bortezomib, erlotinib, gefitinib, imatinib, vemurafenib, and vismodegib), nucleotide analogs and precursor analogs (including, but not limited to, azacitidine, azathioprine, capecitabine, cytarabine, doxifluridine, fluorouracil, gemcitabine, hydroxyurea, mercaptopurine, methotrexate, and tioguanine), peptide antibiotics (including, but not limited to, bleomycin and actinomycin), platinum-based agents (including, but not limited to, carboplatin, cisplatin and oxaliplatin), retinoids (including, but not limited to, tretinoin, alitretinoin, and bexarotene), vinca alkaloids and derivatives (including, but not limited to, vinblastine, vincristine, vindesine, and vinorelbine), or combinations thereof. The chemotherapeutic may comprise a stapled peptide. The chemotherapeutic may in any form necessary for efficacious administration and functionality. “Chemotherapy” designates a therapeutic regimen which includes administration of a “chemotherapeutic” or “anti-cancer drug.”

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

“Peptide stapling” is a term coined from a synthetic methodology wherein two olefin-containing side-chains (e.g., cross-linkable side chains) present in a polypeptide chain are covalently joined (e.g., “stapled together”) using a ring-closing metathesis (RCM) reaction to form a cross-linked ring (see, e.g., Blackwell et al., J. Org. Chem., 66: 5291-5302, 2001; Angew et al., Chem. Int. Ed. 37:3281, 1994). As used herein, the term “peptide stapling” includes the joining of two (e.g., at least one pair of) double bond-containing side-chains, triple bond-containing side-chains, or double bond-containing and triple bond-containing side chain, which may be present in a polypeptide chain, using any number of reaction conditions and/or catalysts to facilitate such a reaction, to provide a singly “stapled” polypeptide. The term “multiply stapled” polypeptides refers to those polypeptides containing more than one individual staple, and may contain two, three, or more independent staples of various spacing. Additionally, the term “peptide stitching,” as used herein, refers to multiple and tandem “stapling” events in a single polypeptide chain to provide a “stitched” (e.g., tandem or multiply stapled) polypeptide, in which two staples, for example, are linked to a common residue. Peptide stitching is disclosed, e.g., in WO 2008/121767 and WO 2010/068684, which are both hereby incorporated by reference in their entirety. In some instances, staples, as used herein, can retain the unsaturated bond or can be reduced. Hydrocarbon stapled polypeptides include one or more tethers (linkages) between two non-natural amino acids, which tether significantly enhances the α-helical secondary structure of the polypeptide. Generally, the tether extends across the length of one or two helical turns (i.e., about 3.4 or about 7 amino acids). Exemplary stapled peptides include those described in U.S. patent Ser. No. 10/259,848, International Patent Application Nos. WO2012/142604 and WO2018106937, each incorporated herein by reference in its entirety.

“Polymersome” refers to a type of artificial vesicles that encloses a solution. The solution within the polymersome and outside the polymersome may be the same or different. Polymersomes are made using amphiphilic synthetic block copolymers to form the vesicle membrane. The copolymer may be, for instance, a diblock or a triblock copolymer. The polymersome membrane provides a physical barrier that isolates the encapsulated material from external materials, such as those found in biological systems. Polymersomes can be generated from double emulsions by known techniques, see Lorenceau et al., 2005, Langmuir 21(20):9183-6, incorporated herein by reference in its entirety.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, the term “target” or “marker” refers to any entity that is capable of specifically binding to a particular targeting moiety. In some embodiments, targets are specifically associated with one or more particular tissue types. In some embodiments, targets are specifically associated with one or more particular cell types. For example, a cell type specific marker is typically expressed at levels at least 2 fold greater in that cell type than in a reference population of cells. In some embodiments, the cell type specific marker is present at levels at least 3 fold, at least 4 fold, at least 5 fold, at least 6 fold, at least 7 fold, at least 8 fold, at least 9 fold, at least 10 fold, at least 50 fold, at least 100 fold, or at least 1000 fold greater than its average expression in a reference population. Detection or measurement of a cell type specific marker may make it possible to distinguish the cell type or types of interest from cells of many, most, or all other types. In some embodiments, a target can comprise a protein, a carbohydrate, a lipid, and/or a nucleic acid.

As used herein, “treat,” “treating” and the like means a slowing, stopping or reversing of progression of a disease or disorder. The term also means a reversing of the progression of such a disease or disorder. As such, “treating” means an application or administration of the methods or agents described herein to a subject, where the subject has a disease or a symptom of a disease, where the purpose is to cure, heal, alleviate, relieve, alter, remedy, ameliorate, improve or affect the disease or symptoms of the disease.

Preferred methods, compositions and materials are described below, although methods, compositions and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### DETAILED DESCRIPTION

Hydrocarbon-stapled peptides are promising tools for disrupting intracellular protein-protein interactions (PPIs). Their primary weaknesses towards clinical translation are (1) their minimal cellular uptake, (2) their lack of cellular targeting, and (3) their solubility. Provided herein is a polymersome based nanocarrier comprising a targeting moiety and configured to induce endosomal escape of stapled peptides into cells.

Experiments conducted during development of embodiments herein exemplified a CD19-targeted nanocarrier to deliver and induce endosomal escape of stapled peptides in human diffuse large B-cell lymphoma (DLBCL). The efficacy, of a pre-clinical stapled peptide, SAH-MS1-18, was dramatically improved by using PEG-SS—PPS polymersomes for cytoplasmic delivery. For delivery into DLBCL cells, the outer surface of PEG-SS—PPS polymersomes was functionalized with CD19-binding Fab antibody fragments, and this facilitated robust uptake and cytoplasmic dissemination into DLBCL cells. While stapled peptides are often only minimally water soluble, encapsulation in PEG-SS—PPS polymersomes allowed for stable solubilization of stapled peptides at concentrations orders of magnitude higher than for the peptides alone (i.e. low mM overall concentrations in polymersome stock solutions). With SAH-MS1-18 as a therapeutic cargo, polymersome delivery improved its therapeutic efficacy by multiple orders of magnitude.

In addition, this nanocarrier platform was used to synergistically exploit two major DLBCL chemoresistance mechanisms, namely p53-inactivation and MCL-1 expression. By therapeutically reactivating p53 in DLBCL using the stapled peptide ATSP-7041, DLBCL cell lines were primed for apoptosis with a specific sensitivity to therapeutic inhibition of MCL-1. While the polymersomes improved the efficacy of the MCL-1 inhibiting stapled peptide by orders of magnitude, priming DLBCL with p53-reactivation made resistant cell lines sensitive and sensitive cell lines more sensitive to MCL-1 inhibition. Few stapled peptides in the literature have been successfully applied in in vivo experiments, and this targeted nanocarrier was able to deliver fluorescent model cargo into human DLBCL cells xenografted in mice.

The present disclosure provides a polymersome comprising a plurality of amphiphilic block co-polymers, a targeting moiety conjugated to a portion of the plurality of the amphiphilic block co-polymers, and an encapsulated cargo molecule. In particular embodiments, the present disclosure provides a polymersome comprising a plurality of amphiphilic disulfide block co-polymers, a targeting moiety conjugated to a portion of the plurality of the amphiphilic disulfide block co-polymers, and an encapsulated cargo molecule, such as a small molecule, peptide, antibody, or stapled peptide. Embodiments herein find particular use in delivering cargo (e.g., therapeutics) with poor in vivo pharmacokinetics (e.g., staple peptides), poor solubility, or problematic toxicity to specific cell types via the targeting moiety on the exterior of the polymersomes.

1. Polymersome

Polymersomes are, like liposomes, a vesicle having membrane which encapsulates an interior solution from an exterior environment. However, the polymersomes are formed amphiphilic non-lipid polymers and the membrane may be a bilayer membrane or a single layer, as in a micelle.

Polymersomes membranes commonly comprise using amphiphilic block copolymers. The copolymer may be, for instance, a diblock or a triblock copolymer. The polymersomes described herein comprise amphiphilic block co-polymers. In some embodiment, the polymersomes described herein comprise amphiphilic disulfide block co-polymers. Amphiphilic disulfide block co-polymers have a disulfide group linking two block copolymers such that the block co-polymer is hydrolyzed in reducing environments. Thus, the block copolymers are reduction sensitive such that when the polymersomes are taken up by the cell, they are disrupted in the endosome. In other embodiments, the polymersomes comprise amphiphilic thioether block co-polymers (see, e.g., Velluto et al. Mol. Pharmaceutics 2008, 5, 4, 632-642; incorporated by reference in its entirety).

The amphiphilic block copolymers comprise at least one of a hydrophilic block and a hydrophobic block. The hydrophilic block may comprise poly(ethylene glycol), poly(ethylene oxide), poly(vinyl alcohol), poly(ethylene-co-vinyl alcohol), poly(N-vinyl pyrrolidone), poly(acrylic acid), poly(ethyloxazoline), poly(alkylacrylates), poly(acrylamides), poly(N-alkylacrylamides), or poly(N,N-dialkylacrylamides). In some embodiments, the hydrophilic block comprises a group consisting of polyglycerols, polyethers, polyethylene glycols, polyesters, polyamides, polyimides, polyimines, polyurethanes, polycarbonates, polyethersulfones, oligopeptides, polypeptides, and copolymers thereof. In some embodiments, the hydrophilic block is a linear polymer. In some embodiments, the hydrophilic block is a branched polymer. The hydrophobic block may comprise poly(propylene sulfide), poly(propylene glycol), esterified poly(acrylic acid), esterified poly(glutamic acid) or esterified poly(aspartic acid).

In some embodiments, the amphiphilic block co-polymers are diblock copolymers comprising poly(ethylene glycol) (PEG) and poly(propylene sulfide) (PPS). In some embodiments, wherein the amphiphilic disulfide block co-polymers are diblock copolymers comprising poly(ethylene glycol) (PEG) and poly(propylene sulfide) (PPS), with an intervening disulfide group separating the hydrophilic PEG from the hydrophobic PPS. In some embodiments, wherein the amphiphilic thioether block co-polymers are diblock copolymers comprising poly(ethylene glycol) (PEG) and poly(propylene sulfide) (PPS), with an intervening thioether group separating the hydrophilic PEG from the hydrophobic PPS. The average molecular weight of the PEG may be between 750 and 1500 Da (e.g., 900-1300 Da, 1000-1500 Da, 1100-1400 Da). In some embodiments, the average molecular weight of the PEG is approximately 1000 Da. In some embodiments, the average molecule weight of the PEG is between 1200 and 1300 Da. The average molecular weight of the PPS may be between 3750 and 4500 Da (e.g., 3800-4500 Da, 3800-4200 Da, 4000-4200 Da). In some embodiments, the average molecular weight of the PPS is approximately 4000 Da.

The ratio of the molecular weights of the block polymers can influence the shape of the type of assembled vesicle, e.g. spherical polymersomes, bicontinuous nanospheres, long wormlike micelles (filomicelles), spherical micelles (see, for example, Allen, S., et al., Journal of Controlled Release 262, 91-103 (2017), incorporated herein by reference in its entirety). Any ratio of molecular weights may be used that allow or facilitate formation of polymersomes.

The polymersomes are on average about 100-150 nm. In some embodiments, the hydrodynamic radius of the polymersomes are between 50 and 150 nm. In some embodiments, the polymersomes are micelles with a hydrodynamic radius between 10 and 50 nm. The size of the polymersome may vary with the methods of making and the type and quantity of the cargo molecule.

Block co-polymers and components thereof are described, for example, in U.S. Pat. No. 10,335,499; incorporated by reference in its entirety.

2. Targeting Moiety

The polymersome described herein comprise a targeting moiety conjugated to the exterior or outer membrane surface of the polymersome. A targeting moiety refers to any moiety that binds to a component of a cell. Such a component is referred to as a “target” or a “marker.” Typically, the binding of a targeting moiety to a component of a cell will be a high affinity binding interaction such that the targeting moiety is specifically binding cells comprising a particular target associated with a particular organ, tissue, cell, and/or subcellular locale. The target may be any cellular component that is exclusively or primarily associated with one or a few cell types, with one or a few diseases, and/or with one or a few developmental stages.

The targeting moiety may be a polypeptide, glycoprotein, nucleic acid, small molecule, carbohydrate, lipid, etc. For example, a targeting moiety can be a nucleic acid targeting moiety (e.g. an aptamer, Spiegelmer®, etc.) that binds to a cell type specific marker. In general, an aptamer is an oligonucleotide (e.g., DNA, RNA, or an analog or derivative thereof) that binds to a particular target, such as a polypeptide. The targeting moiety may be a naturally occurring or synthetic ligand for a cell surface receptor, e.g., a growth factor, hormone, LDL, transferrin, etc. The targeting moiety may be an antibody or any characteristic fragment thereof. Synthetic binding proteins such as Affibodies®, Nanobodies™, AdNectins™, Avimers™, etc., may be used. Peptide and non-antibody protein targeting moieties can be identified, e.g., using procedures such as phage display (e.g., RGD peptides, NGR peptide, and transferrin LHRH). This widely used technique has been used to identify cell specific ligands for a variety of different cell types. The small molecules may include synthetic or natural molecules which target specific receptors or binding partners (e.g., folate, galactose).

In some embodiments, the targeting moiety is an antibody or antibody fragment. In some embodiments, such antibodies are monoclonal. In some embodiments, the antibody or antibody fragments recognize or bind to markers or tumor-associated antigens that are expressed at high levels on target cells and that are expressed predominantly or only on diseased cells versus normal tissues, and antibodies that internalize rapidly. Antibodies useful within the scope of the present invention include antibodies or antibody fragments (e.g., mAbs) include, but are not limited to, in cancer: LL1 (anti-CD74), LL2 (anti-CD22), RS7 (anti-epithelial glycoprotein-1 (EGP-1)), PAM-4 and KC4 (both anti-MUC1), MN-14 (anti-carcinoembryonic antigen (CEA, also known as CD66e), Mu-9 (anti-colon-specific antigen-p), Immu 31 (an anti-alpha-fetoprotein), TAG-72 (e.g., CC49), Tn, J591 (anti-PSMA (prostate-specific membrane antigen)), G250 (an anti-carbonic anhydrase IX mAb) and L243 (anti-HLA-DR). In some embodiments, targeting moieties comprise antibodies that recognize/bind to HER-2/neu, BrE3, CD19, CD20 (e.g., C2B8, hA20, 1F5 Mabs) CD21, CD23, CD80, alpha-fetoprotein (AFP), VEGF, EGF receptor, P1GF, MUC1, MUC2, MUC3, MUC4, PSMA, gangliosides, HCG, EGP-2 (e.g., 17-1A), CD37, HLA-DR, CD30, Ia, A3, A33, Ep-CAM, KS-1, Le(y), 5100, PSA (prostate-specific antigen), tenascin, folate receptor, Thomas-Friedenreich antigens, tumor necrosis antigens, tumor angiogenesis antigens, Ga 733, IL-2, IL-6, T101, MAGE, antigen to which L243 binds, CD66 antigens, i.e. CD66a-d or a combination thereof.

In some embodiments, an antibody targeting moiety is a human antibody or a humanized antibody.

In some embodiments, the targeting moiety binds a protein expressed on the surface of a cell. In some embodiments, the targeting moiety binds the CD19 protein.

In some embodiments, the targeting moiety comprises the Fc portion of an immunoglobulin. In some embodiments, the targeting moiety comprises the Fc portion of an IgG. In some embodiments, the Fc portion of an immunoglobulin is a human Fc portion of an immunoglobulin. In some embodiments, the Fc portion of an IgG is a human Fc portion of an IgG.

In some embodiments, the targeting moiety is covalently bound to the block copolymer. In some embodiments, the targeting moiety is bound to the hydrophilic polymer. In other embodiments, the targeting moiety is associated with the polymersome by non-covalent bonding interactions such as ionic or by van der Waals forces.

In some embodiments, a portion of the amphiphilic block copolymers comprise a functional group to which the targeting moiety may be conjugated. Functional group pairs are well-known in the art and suitable for use with the polymersomes described herein. In some embodiments, The targeting moiety may comprise a cysteine linker to facilitate conjugation to at least a portion of the amphiphilic block copolymers which comprise a functional group that facilitates cysteine or thiol conjugation reactions (e.g. an azide). The linkers may comprise any amino acid sequence. The linkers may be flexible such that they do not constrain either of the two components they link together in any particular orientation. Other embodiments for conjugation of the targeting moiety to the polymersome are within the scope herein. For example, other ‘click’ chemistries are available for such conjugations. In some embodiments, the targeting moiety is fused to an affinity protein (e.g., streptavidin, HALOTAG, etc.) and the polymersome displays a complementary affinity molecule (e.g., biotin, a haloalkane, etc.).

The polymersome may comprise a number of targeting moieties conjugated to the plurality of amphiphilic disulfide block co-polymers. In some embodiments, the targeting moiety is conjugated to less than 1% of the amphiphilic disulfide block co-polymers. The targeting moiety may be conjugated to 0.01-1% of the amphiphilic disulfide block co-polymers. In some embodiments, the targeting moiety is conjugated to about 1%, about 0.75%, about 0.5% about 0.25%, about 0.1%, about 0.05%, or about 0.01% of the amphiphilic disulfide block co-polymers. In some embodiments, the targeting moiety is conjugated to about 0.01-0.75%, about 0.01-0.5%, about 0.01-0.25%, about 0.01-0.1%, about 0.01-0.05%, about 0.05-1%, 0.05-0.75%, about 0.05-0.5%, about 0.05-0.25%, about 0.05-0.1%, about 0.1-0.05%, about 0.1-1%, 0.1-0.75%, about 0.1-0.5%, about 0.1-0.25%, about 0.25-1%, 0.25-0.75%, about 0.25-0.5%, about 0.5-1%, 0.5-0.75%, or 0.75-1% of the amphiphilic disulfide block co-polymers.

3. Cargo Molecule

The polymersome comprises an encapsulated cargo molecule. The cargo molecule may comprise a therapeutic agent, a marker, or a combination thereof.

The marker may comprise a contrast agent and dye for visualization within a cell (e.g. fluorescent dyes). Suitable fluorescent dyes include, but are not limited to: xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phthalocyanine, bilirubin, etc.), CF dye (Biotium), BODIPY (Invitrogen), ALEXA FLOUR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc.

The therapeutic agent refers to any drug, pharmaceutical substance, or bioactive agent which treats and/or cures a disease or disorder (e.g., a cancer). In particular embodiments, the agent exhibits poor pharmacokinetics and/or is toxic when administered in vivo. In some embodiments, the present technology allows for delivery of the agent to target cells, overcoming the limitations inherent to the agent itself. The therapeutic agent may comprise therapeutically useful peptides, polypeptides, polynucleotides, and other therapeutic macromolecules as well as small molecule and/or synthetic pharmaceuticals or drugs. In some embodiments, the cargo molecule is a small molecule drug. In some embodiments, the cargo molecule is a hydrophobic small molecule drug. The cargo molecule may comprise a single therapeutic agent or multiple types of therapeutic agents (e.g., a stapled peptide and a small molecule) or multiple therapeutic agents of a single type (e.g., two types of stapled peptides or two types of small molecules).

In some embodiments, the cargo molecule comprises a peptide. In some embodiments, the cargo molecule comprises a stapled peptide. In some embodiments, the cargo molecule comprises a hydrocarbon stapled peptide. In some embodiments, the cargo molecule comprises a hydrophobic stapled peptide. In some embodiments, the stapled peptide comprises polar and/or charged side chains (e.g. pre-clinical stapled peptides). The stapled peptide may be an inhibitor of protein-protein interactions. In some embodiments, the cargo molecule comprises more than one type of stapled peptide (e.g., two stapled peptides with two different protein-protein interaction targets).

The present disclosure also provides a composition comprising the polymersomes described herein and a carrier. In some embodiments, the composition comprises a single type of polymersome encapsulating a single type of cargo molecule (e.g. stapled peptide). In some embodiments, the composition comprises a single type of polymersome encapsulating more than one type of cargo molecule (e.g., a stapled peptide and a small molecule drug or a stapled peptide and a marker). In some embodiments, the composition comprises more than one type of polymersome as described herein individually encapsulating one or more types of cargo molecules.

Carriers, also referred to as excipients, may include any and all solvents, dispersion media, coatings, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as preservatives and antioxidants. The compositions of the present invention and methods for their preparation will be readily apparent to those skilled in the art.

The disclosed compounds may be incorporated into pharmaceutical compositions suitable for administration to a subject (such as a patient, which may be a human or non-human). The pharmaceutical compositions may include a “therapeutically effective amount” of the cargo molecule. A “therapeutically effective amount” refers to an amount effective, at dosages (single dose or part of a series) and for periods of time necessary, to achieve the desired therapeutic result. A therapeutically effective amount may be determined by a person skilled in the art and may vary according to factors such as the disease state, age, sex, and weight of the individual, and the ability of the composition to elicit a desired response in the individual. A therapeutically effective amount is also one in which any toxic or detrimental effects are outweighed by the therapeutically beneficial effects.

The compositions may be formulated for any appropriate manner of administration, and thus administered, including for example, oral, intravenous, epicutaneous, intradermal, intraperitoneal, subcutaneous, or intramuscular administration. In some embodiments, the polymersomes or compositions thereof are “administered parenterally,” usually by injection, including, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Techniques and formulations may generally be found in “Remington's Pharmaceutical Sciences,” (Meade Publishing Co., Easton, Pa.). Therapeutic compositions must typically be sterile and stable under the conditions of manufacture and storage. The route or administration may dictate the type of carrier to be used.

The present disclosure further provides methods of treating a disease or disorder comprising administration of a therapeutically effective amount of the polymersome or polymersome compositions described herein.

Disorders in which a patient would benefit from treatment with the dosage forms disclosed herein may include those which associated with protein-protein interactions which can be disrupted by the use of stapled peptides, including but not limited to cancer, neurological and neurodegenerative diseases, infectious diseases, and hormonal regulation and endocrine disorders (See, for example Ali et al. Structural Biotechnology Journal 17 (2019) 263-281, incorporated herein by reference in its entirety).

In some embodiments, the disease or disorder is cancer. The abnormal regulation of protein-protein interactions contributes to the majority of cancers due to their involvement in all phases of oncogenesis, from cell proliferation, cell survival, and inflammation to invasion and metastasis The polymersomes described herein or composition thereof may be used to treat any cancer type or subtype. The cancer may be a carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, muscle tissue, ovary, pancreas, prostate, skin, spleen, stomach, testicle, thyroid or uterus. In some embodiments, the cancer is a solid tumor. In some embodiments, the cancer is human diffuse large B-cell lymphoma (DLBCL). For cancer treatment, the polymersomes described herein, or compositions thereof may be administered locally to the cancer, such as intratumoral.

A wide range of second therapies may be used in conjunction with the compounds of the present disclosure. The second therapy may be a combination of a second therapeutic agent or may be a second therapy not connected to administration of another agent. Such second therapies include, but are not limited to, surgery, immunotherapy, radiotherapy, or administration of a second chemotherapeutic agent.

### EXAMPLES

### Materials and Methods

Synthesis of PPS-PDS: poly(propylene sulfide)(PPS) with pyridyl disulfide (PDS) end-group (compound 1) Thiol-functionalized PEGs were purchased from Laysan Bio Inc. (mPEG-SH) and Nanosoft Polymers (N3-PEG-SH) and used as delivered. Both PEGs were advertised with MW 1,000 Da, though by NMR and MALDI measurements were approximately 1,200 Da, and PPS degree of polymerization (DP) was scaled accordingly to maintain previously reported block ratios.

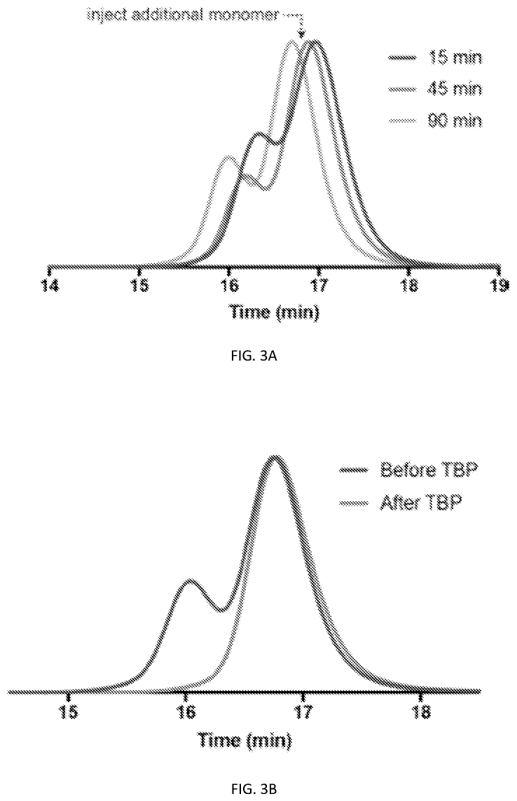

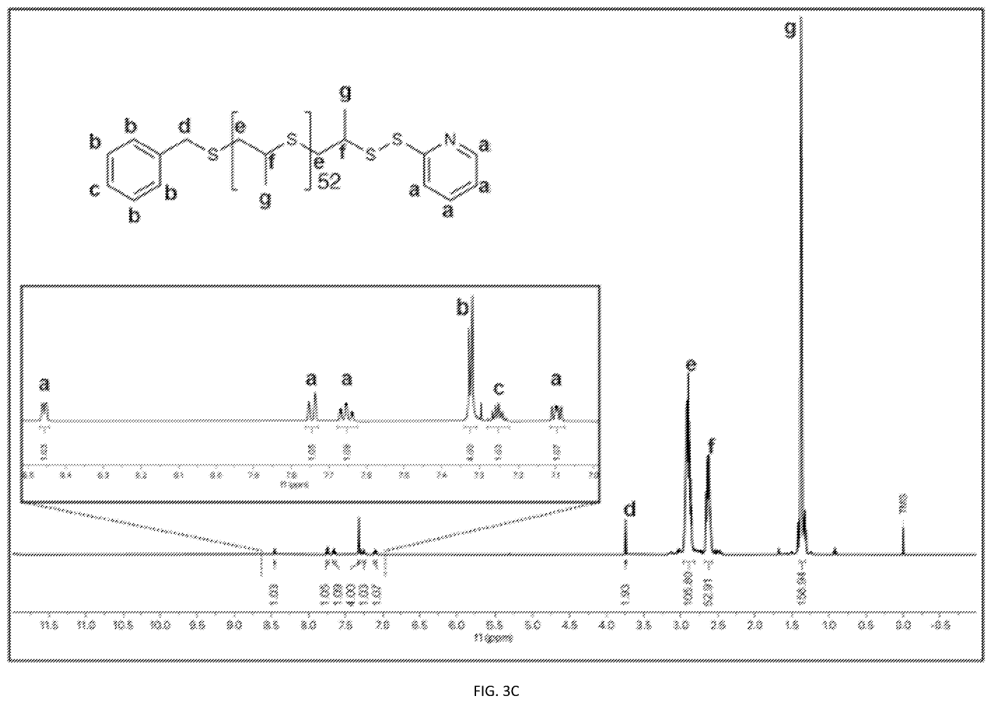

Benzyl mercaptan (1 eq.) in degassed, anhydrous THF (20 mM) was deprotonated with sodium methoxide (NaOMe; 1.1 eq.) under nitrogen protection for 30 minutes. Propylene sulfide (53.3 eq.) was rapidly added by syringe under vigorous stirring and nitrogen protection. The reaction was carried out under a constant flow of vented nitrogen protection to prevent pressure accumulation. The reaction proceeded to completion within 1 hour, according to 1H NMR, at which point the thiolates were quenched with acetic acid (AcOH; 2 eq.). Disulfide-dimerized PPS chains were then reduced by adding triethylamine (TEA; 3 eq.), water (H2O; 8 eq.), and tributylphosphine (TBP; 8 eq.) under nitrogen protection for four hours. Aldrithiol-2 (25 eq.) was dissolved in a minimal amount of THF and degassed, and the PPS reaction mixture was cannulated dropwise into the capping solution under nitrogen protection and vigorous stirring and stirred overnight. THF was then removed, and the crude yellow oil was extracted with methanol repeatedly until clear. The removal of aldrithiol-2 and the mercaptopyridine byproduct were confirmed by silica TLC with a mobile phase of 2% methanol in DCM. The fluorescence indicator under UV light was used to detect aldrithiol-2 and mercaptopyridine. CAM staining was used to detect PPS-PDS. Dragendorff staining was used to detect mercaptopyridine. The pure PPS-PDS was dried under high vacuum, and the final product was a clear oil with a slight yellow tint. Purity was confirmed by DMF GPC, NMR (FIG. 3), and TLC. Compound 1 was stored under argon protection at −80° C.

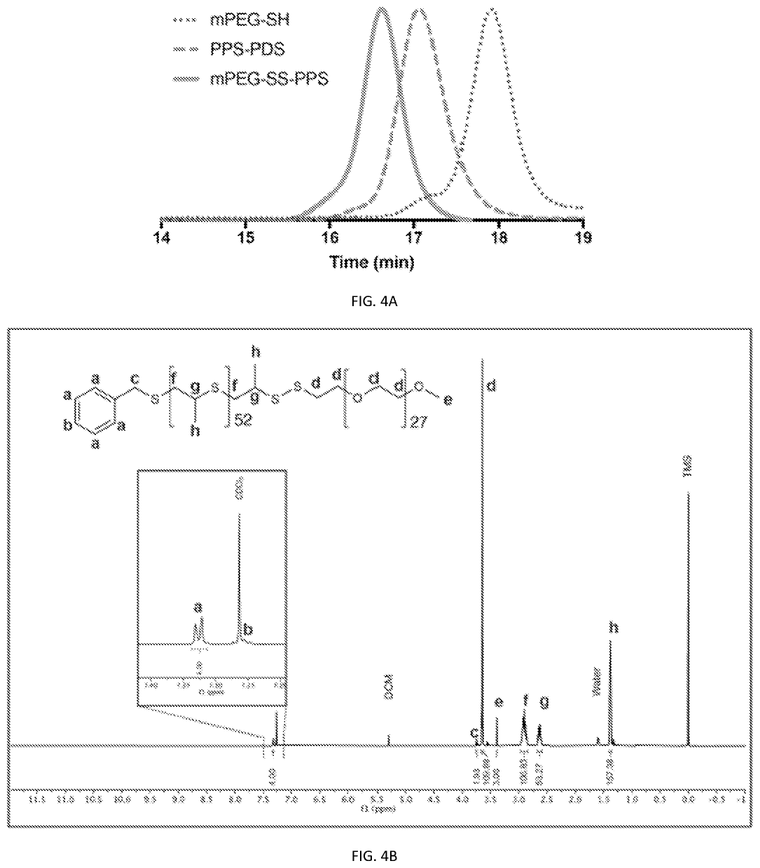

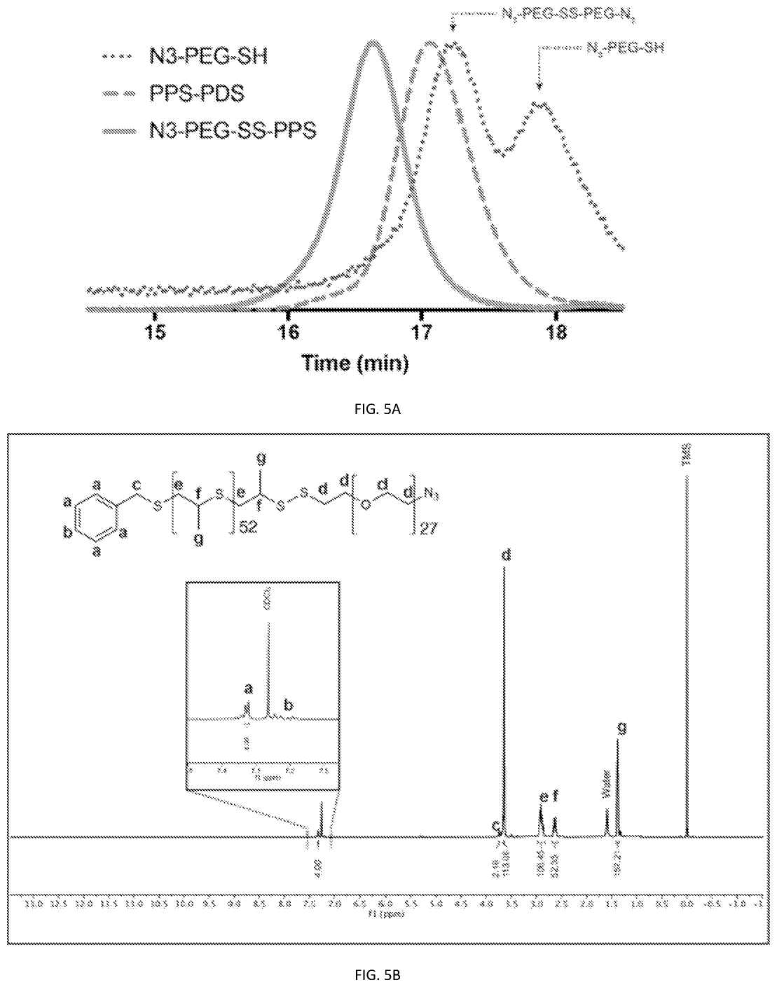

Synthesis of methoxy- and azide-poly(ethylene glycol)-block-poly(propylene sulfide) (mPEG-SS—PPS (compound 2) and N₃-PEG-SS—PPS (compound 3)) PPS-PDS (1.2 eq. PDS) and R-PEG-SH (R=OMe or N3; 1 eq. free thiol (as determined by polymer mass and dimerization degree by GPC) were each dissolved in DCM (1 g/mL and 0.01 g/mL respectively) and degassed under nitrogen bubbling. The PEG solution was cannulated dropwise into the PPS solution under vigorous stirring and allowed to react overnight, and the reaction mixture gradually turned yellow. The crude product was concentrated and purified over a gradient silica flash column. 30 grams of dry silica per gram of crude mixture (assuming no solvent) was loaded into a flash column as a slurry in DCM. The concentrated sample was loaded onto the column in DCM, in which there was very little migration. The column was then washed with 2% methanol in DCM, in which PPS-PDS and PPS—PPS disulfides washed off the column. Due to the refractive index matching of the silica and solvent, this migration was visible by eye as an opaque band. The yellow mercaptopyridine byproduct also visibly eluted in this washing step. The PEG-SS—PPS band, still visible at the top of the column, was then eluted with 10% methanol in DCM. Behind the eluting band, the silica visibly turned opaque as the methanol saturated the silica. The solvent from the eluted product was then removed by rotary evaporation. A minimal amount of DMF was used to transfer the polymer to 50 mL centrifuge tubes. The polymer was precipitated with −20° C. MeOH at a ratio of 1:10 or greater and centrifuged at 4,700 g at −10° C. until the supernatant was visibly clear. The deceleration rate was minimized to avoid disturbing the oil when the centrifuge stopped. The clear supernatant was decanted, and the oil was then extracted two more times with −20° C. MeOH, centrifugation, and decanting. After the MeOH extractions, removal of DMF and co-eluting PEG was confirmed by NMR and by TLC with CAM staining and a mobile phase of 8% methanol in DCM. The polymer was then redissolved in DCM, filtered through a 0.2 μm filter into pre-weighed scintillation vials, and immediately dried by rotary evaporation followed by high vacuum. The final product was a clear, slightly yellow oil, confirmed pure by DMF GPC, NMR (FIGS. 4 and 5), and TLC. All polymers were stored under argon protection at −80° C.

In initial syntheses, the heterodisulfides were synthesized in the opposite direction, by first making PEG-PDS and reacting it with PPS—SH. However, the method described above allowed for a simpler, more effective workup and a stable, capped PPS intermediate. Therefore, the scaled-up syntheses were done as presented above, though both synthesis routes produced indistinguishable final products.

Synthesis and purification of hydrocarbon-stapled peptides For peptide synthesis, rink amide AM low loading resin was purchased from Sigma Aldrich (8.55120). Solvents and natural amino acids were purchased from Gyros Protein Technologies, while stapling amino acids were purchased from Sigma Aldrich or Advanced ChemTech.

All-hydrocarbon stapled peptides were synthesized on a PreludeX peptide synthesizer from Gyros Protein Technologies, primarily using chemistries described previously. (Ref. 46; incorporated by reference in its entirety). First, the resin was swelled in DCM for 15 min followed by DMF for 15 min. Deprotection reactions were done with 20% piperidine in NMP for 2×10 min, with the exception of stapling amino acids, which were deprotected for 4×10 min. Of note, because the α-carbon of the stapling amino acids is di-substituted, their N-termini fail to generate a purple Kaiser test, even when they are successfully deprotected. Unless otherwise specified, coupling reactions used 10 eq. of amino acid (300 mM solution in NMP), 9.5 eq of HATU (285 mM solution in NMP), and 20 eq of DIPEA (600 mM solution in NMP) for 30 min. Stapling amino acids were coupled using half the amount of each solution for 1 hr. To couple the amino acid directly following a stapling amino acid, the coupling reaction was repeated for 4×1 hr, except Cba, which was repeated for 2×4 hr. For very large scale synthesis of SAH-MS1-18, double coupling with 5 eq. amino acid was used for regular amino acids, and longer reaction times with 5 eq. amino acid were used for the positions after S5 and R8. After each coupling reaction, the resin was exposed to capping solution (4/1/0.1 NMP/Ac2O/DIPEA) for 10 min to cap any unreacted amines, generate truncation impurities instead of deletion impurities, and simplify HPLC purification. After every reaction step, the resin was washed with alternating washes of DMF and DCM.

After completing the linear synthesis, peptides to be acetylated were deprotected and capped with capping solution. For FITC-labeled peptides, the N-terminal beta-alanine remained FMOC-protected during the RCM reaction. For RCM stapling, the resin was washed thoroughly with DCM, then suspended in a 4 mg/mL solution of Grubbs 1st generation catalyst in anhydrous 1,2-dichloroethane with 20 mol % catalyst with respect to resin substitution. The catalyst solution was prepared fresh immediately before stapling. The stapling reaction was carried out under nitrogen bubbling for cycles of 3×2 hr followed by 3×4 hr, with DCM washing between cycles. Stapling was confirmed by LCMS through the loss of ethylene (28 Da). For FITC-labeled peptides, the resin was then deprotected and reacted with 300 mM FITC, isomer I (Sigma Aldrich, F7250) and 600 mM DIPEA for overnight or longer. FITC-conjugation was confirmed with LCMS.

Completed peptides were then cleaved from the resin. The resin was washed thoroughly with DCM and dried, followed by TFA cleavage using a fresh solution of 95/2.5/2.5 TFA/H₂O/TIS for 2 hours. After the TFA solution was collected, the resin was washed once with TFA solution, the TFA solutions pooled, and the peptide immediately precipitated using 50/50 hexane/diethyl ether in 50 mL centrifuge tubes at a volume ratio of 10:1 or greater. The solution was chilled at −80° C. for 1 hour, then the peptide was pelleted by centrifugation at 1,500 g for 20 min at −10° C. The crude pellet was dried, resuspended in an H₂O/ACN mixture, and lyophilized. The peptide was then resuspended in a minimum volume 50/50 H₂O/ACN with ammonium bicarbonate buffer at roughly neutral pH and allowed to sit at room temperature at least overnight. This facilitated the complete deprotection of the carbamic acid on tryptophan side chains, as identified by MW+44 impurities in LCMS (Ref. 46; incorporated by reference in its entirety). Complete deprotection of ATSP-7041 proceeded slowly, and the peptide began to precipitate after a few hours. A large quantity or urea was dissolved into the solution and sonicated, which redissolved the peptide.

The peptide solutions were then filtered and purified via reverse-phase HPLC-MS using a C18 column from Waters (XBridge Peptide BEH C18, 130° A, 5 μm, 19 mm×150 mm) with mobile phases A (water+0.1% formic acid) and B (ACN) unless otherwise noted. The pure fractions were pooled, concentrated by rotary evaporation, and lyophilized. The peptides were redissolved in 30% ACN in H₂O, filtered, aliquoted, lyophilized, confirmed pure by LCMS, and quantified by amino acid analysis (AAA; UC Davis Molecular Structure Facility).

SAH-MS1-18 had poor chromatographic shape and inconsistent retention times with formic acid as the mobile phase modifier. Instead, 0.10% TFA was added to both A and B mobile phases for this peptide, which improved the chromatography significantly. After the purified peptide was lyophilized, it was dissolved in a minimal amount of glacial acetic acid with a small amount of water and acetonitrile for complete dissolution. After a few minutes, the solution was diluted with Milli-Q water, re-lyophilized, then aliquoted and analyzed as described above.

Reverse-phase liquid-chromatography mass-spectrometry (LCMS) analysis of peptides LCMS was used to confirm the completion of synthesis reactions, measure peptide purity, and measure peptide concentrations in polymersome formulations. An analytical column was used to match the purification column and facilitate method transfer (XBridge Peptide BEH C18, 130° A, 5 μm, 4 mm×150 mm) with mobile phases A (water+0.1% TFA) and B (ACN). An example of a general method includes a 5 minute isocratic loading phase at 10% B, a 3-5%/minute separation gradient, then a column wash at 100% B for 5-10 minutes, followed by re-equilibration at 10% B. Columns were always stored in 100% acetonitrile. All samples were filtered through a 0.2 μm filter, except polymersomes that had been extruded, which should also remove dust and debris. Peptide purity was calculated by the Agilent software integrating the 220 nm absorbance chromatogram.

Peptide concentration in polymersomes was measured by running a AAA-quantified standard sample and using the area under the curve of the peptide peak's UV absorbance to calculate the amount of peptide injected from an unknown sample. The area under the curve is directly proportional to the amount of peptide injected. The polymer seemed to interact strongly with the column, so after a set of polymersome samples, the column was washed with acetonitrile, DCM, then acetonitrile again, being careful to never have water and DCM in the column at the same time.

Polymersome assembly For thin film assembly, the polymers were dissolved in DCM, and 10 mg of polymer was transferred to a 2 mL glass vial that had first been piranha-etched. The DCM was evaporated under high vacuum to form a thin layer of polymer film on the glass walls. 250 μL of sterile PBS was added to the vial, and the vial was slowly rotated at room temperature for 2-3 days, until no polymer was visible on the vial walls.

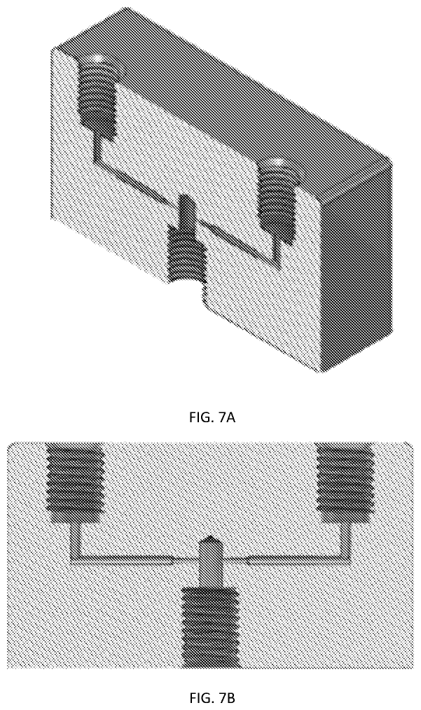

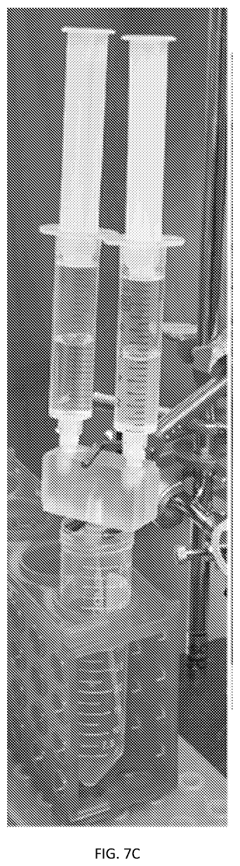

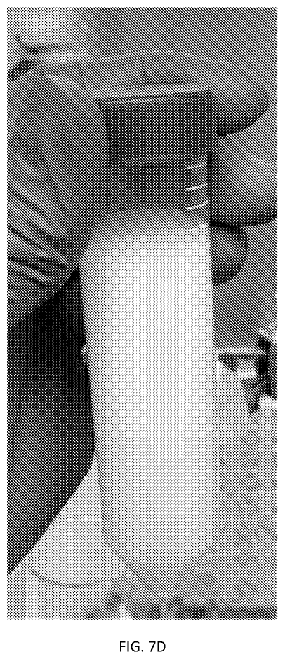

For flash nanoprecipitation (FNP), a CIJ-D device (FIG. 7) was 3D-printed using the same design parameters originally reported previously (Ref. 50; incorporated by reference in its entirety) and previously used by others for assembly of PEG-PPS polymersomes (Refs. 48-49; incorporated by reference in their entireties). 3D-printing allowed for rapid, reproducible assembly of these devices. Syringe adapters (IDEX P604) and outlet adapters (IDEX P202X, IDEX P200X) were purchased from Fisher Scientific. The outlet tubing used was 1/16″ O.D. and 0.04″ I.D. Before each use, the device was sterilized and cleaned with 0.5 M NaOH and rinsed repeatedly with Milli-Q water. All assemblies were done in a sterile hood, following the protocols and ratios previously described previously (Refs. 48-49; incorporated by reference in their entireties).

For calcein encapsulations, a 100 mM calcein solution was prepared at physiological osmolarity (˜313 mOsm). Calcein in its protonated form (Calcein High Purity, Thermo Fisher Scientific) was first dissolved in 2 molar equivalents of NaOH from a 1 M solution, then 13 mOsm worth of 1×PBS, pH 7.4 (Gibco, Thermo Fisher Scientific), was added. The solution was then diluted to a final calcein concentration of 100 mM using Milli-Q water for a final osmolarity of 313 mOsm. This solution was used both as the anti-solvent stream in the syringe and as the dilution reservoir during FNP encapsulation.

For stapled peptide encapsulations, polymer was dissolved in THF at 40-100 mg/mL. SAH-MS1-18 or BIM-SAHB was added from a DMSO stock solution (20-100 mM) at peptide:polymer mass ratios of ˜1:4, then this THF solution was diluted 1:1 with PBS in an attempt to solubilize as much peptide as possible. This solution was then impinged against PBS into a PBS reservoir. For the largest-scale assemblies, the PBS-dilution step was omitted, and THF was removed from the FNP-mixed solution by rotary evaporation to make a highly concentrated polymersome solution.

All polymersome samples were then extruded 11-21 times through a 100 nm pore-size membrane (Whatman Nucleopore Track-Etched Membrane, 19 mm, 100 nm) using a syringe-driven Mini Extruder (Avanti Polar Lipids) in a sterile hood. Size and dispersity were monitored by DLS. The polymersomes were then immediately purified from any residual organic solvents using gravity-driven disposable PD-10 desalting columns containing Sephadex G-25 resin (GE Healthcare) into 1×PBS, pH 7.4 (Gibco, Thermo Fisher Scientific). If non-encapsulated cargoes needed to be fully removed and no further workup would be performed, then the polymersomes were instead purified into PBS over Sepharose CL-4B or using a 300 kDa MWCO MicroKros device to fully remove non-encapsulated cargoes.

Measuring polymersome stability in serum via calcein fluorescence dequenching Polymersomes encapsulating a self-quenching calcein solution were assembled as described above. The resulting stock solution was diluted 1:100 into either RPMI 1640 (“media”), media+10% fetal bovine serum (FBS), or media+10% FBS+5 mM Triton X-100 in a black, flat-bottom 96-well plate. Samples were incubated at 37° C., and the calcein fluorescence was monitored for 1 hour via plate reader (SpectraMax iD5, Molecular Devices). Each sample was prepared in quadruplicate, and each value was background-subtracted using corresponding samples prepared by diluting pure PBS instead of polymersomes into the indicated solution (though all background solutions had negligible fluorescence values).

Aqueous size-exclusion high-performance liquid chromatography (SEC HPLC) The same PBS solution was used as the mobile phase as for polymersome assembly and for dissolving lyophilized peptides before SEC HPLC. The column used was AdvanceBio SEC, 130 Å, 2.7 μm, 4.6 mm diameter with a 50 mm length guard column in series with a 150 mm column (Agilent). The polymersome solution was stored at 4° C. for one month before analysis. Peptide concentrations in the polymersome solution were measured by LCMS using the area under the curve of the UV absorbance chromatogram, and SEC HPLC samples injected were equimolar in peptide as measured by reverse-phase LCMS.

Fab design The αCD19 Fab was designed using published variable region sequences (Vκ and VH) from HD37 mouse-anti-human-CD19 IgG (Refs. 54-55; incorporated by reference in their entireties), for both light chain (GenBank CAA67620, amino acids 1-111) and heavy chain (GenBank CAA67618, amino acids 1-124), combined with constant regions (C_{κ} and C_{H}) from mouse IgG consensus sequences for light chain (UniProt P01837, amino acids 1-107) and heavy chain (UniProt P01868, amino acids 1-104). To create an irrelevant control Fab, the variable regions were substituted for those from an antibody specific for xenoantigen OspA without changing the constant regions (Refs. 56-57; incorporated by reference in their entireties). A cysteine linker ( . . . GSGGSSGSGC) was encoded on the C-terminus of the heavy chain to create αCD19-cys and αOspA-cys for site-specific conjugation to polymersomes.

Fab cloning Fab sequences were acquired as gBlocks Gene Fragments (Integrated DNA Technologies) and cloned into an AbVec2.0 plasmid under a cytomegalovirus (CMV) promoter for constitutive mammalian expression (Ref. 64; incorporated by reference in its entirety). A signal peptide sequence derived from osteonectin was added to the N-terminus of both light and heavy chains to induce protein secretion. The plasmid also contained an ampicillin resistance gene under a constitutive E. coli promoter. After cloning and transformation into competent DH5α, the plasmid was selected for using ampicillin, and propagated by bacterial growth in lysogeny broth (LB) with 100 μg/mL ampicillin in shaker flasks at 37° C. The plasmid was isolated using NuelcoBond Xtra Maxi kits (Machery Nagel). Purified plasmids were sequenced at the University of Chicago Comprehensive Cancer Center DNA Sequencing and Genotyping Facility (UCCCC-DSF), and all sequences were confirmed to align with the designed sequences (Benchling).

Fab expression and purification Fabs were expressed in HEK293T suspension cells in FreeStyle 293 Expression Medium (Thermo Fisher Scientific). At 1 million cells/mL in log-phase growth, cells were transfected with 1 μg of plasmid and 2 μg of polyethyleneimine in 40 μL OptiPRO SFM (Gibco) per million cells. Transfected cells were cultured for 6 days in shake flasks at 37° C. and 5% CO2. The cells were then pelleted by centrifugation, and the supernatant was filtered through a 0.22 μm filter and pH-adjusted to 7.0 using 1 M Tris buffer, pH 9.0. The Fabs were then purified by affinity chromatography using 5 mL HiTrap Protein G HP columns (GE Life Sciences) via fast protein liquid chromatography (AKTA FPLC, GE Healthcare). A dedicated column was used for each Fab to prevent cross-contamination. For large scale purification, up to 3×5 mL columns were connected in series. The column was first equilibrated with 5 column volumes (CVs) of PBS at 5 mL/min. The crude Fab solution was then flowed over the column at 5 mL/min and the column washed with 10 CVs of PBS. Pure Fab was eluted with 0.1 M glycine-HCl, pH 2.7, into 3 mL fractions pre-buffered with 125 μL of 1 M Tris buffer, pH 9.0, and 1 mL of 1×PBS, pH 7.4, to achieve a neutral pH in each fraction upon elution. The crude flow-through was collected and the purification repeated multiple times until the UV-absorbance of the elution peak was minimal. Elution peaks were pooled, dialyzed extensively (Slide-A-Lyzer, G2 Dialysis Cassettes, 10 kDa MWCO, Thermo Fisher Scientific) against 1×PBS, pH 7.4, concentrated (Amicon Ultra-15, 10 kDa MWCO, Millipore Sigma) to no more than 10 mg/mL, sterile filtered, and either stored at 4° C. or aliquoted and frozen for later use.

Fab concentrations were calculated using UV absorbance based on their calculated extinction coefficients at 280 nm (48,923 M⁻¹ cm⁻¹ for αCD19-cys and 47,432 M⁻¹ cm⁻¹ for αOspA-cys).

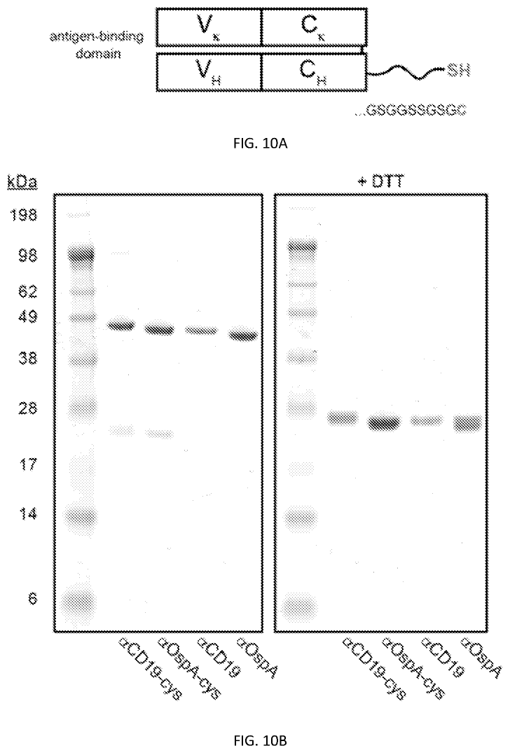

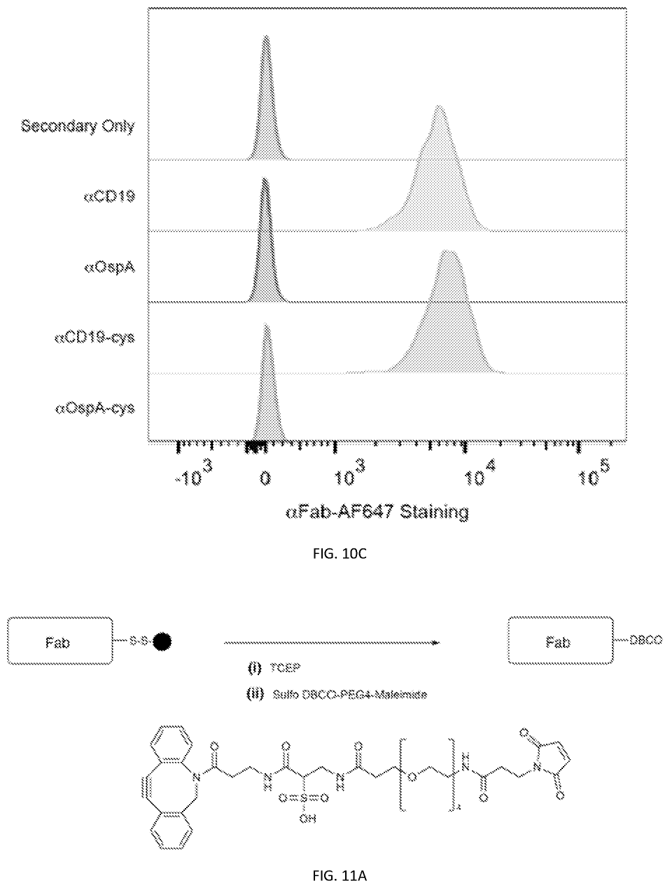

FAB functionalization with DBCO Coomassie-stained SDS-PAGE was used to determine the fraction of each sample that was unimeric, intact Fab, as opposed to Fab-Fab disulfides or free heavy/light chain, which were the two other minor bands in some samples (FIG. 10). The fraction of intact, unimeric Fab was always >80%. The concentration of unimeric, intact Fab, was then calculated as the product of the concentration determined by UV absorbance at 280 nm and the fraction determined by SDS-PAGE.

Before the reduction reaction, EDTA (UltraPure, 0.5 M EDTA, pH 8.0; Invitrogen) was added to a final concentration of 10 mM to the Fabs in PBS, pH 7.4. TCEP, aliquoted in Milli-Q water and frozen at 1 M, was diluted immediately before use to 1 mM in PBS+10 mM EDTA, pH 7.4. TCEP (0.85 equivalents with respect to the concentration of intact, unimeric Fab) was added to the Fab, and the reaction was immediately vortexed. The reaction was incubated at 37° C. for 90 minutes. The heterobifunctional linker, Sulfo-DBCO-PEG4-Maleimide (Click Chemistry Tools), was dissolved immediately before use at 20 mM in PBS with 10 mM EDTA, pH 7.4. 100 equivalents of the linker were added to the reduced Fab without workup, and the reaction was immediately vortexed and incubated at room temperature for 1 hour. After 1 hour, the Fab was immediately purified by 8 rounds of diafiltration into 1×PBS, pH 7.4, at 4° C., using Amicon ultrafiltration devices with a 10 kDa MWCO and a volume appropriate to the scale of the reaction to avoid concentrating the Fabs to greater than 10 mg/mL. Functionalized Fabs were then sterile filtered.

After purification of Fab-DBCO, the Fab concentration was calculated using equation 4.1:

$$Concentration of Fab ( M ) = \frac{{A}_{280} - ( {A}_{309} \times CF )}{{ϵ}_{Fab , 280}} \qquad ( 4.1 )$$

with A₂₈₀ and A₃₀₉ the sample absorbance at 280 nm and 309 nm, respectively, the correction factor

$$CF = \frac{{ϵ}_{DBCO , 280}}{{ϵ}_{DBCO , 309}} = 1.089 ,$$

and ^{∈}Fab, 280, the extinction coefficient of the Fab at 280 nm (48,923 M⁻¹ cm⁻¹ for αCD19-cys and 47,432 M⁻¹ cm⁻¹ for αOspA-cys).

DBCO concentration was calculated using equation 4.2:

$$Concentration of DBCO ( M ) = \frac{{A}_{309}}{{ϵ}_{DBCO , 309}} \qquad ( 4.2 )$$

with ^{∈}DBCO, 309=12,000 M⁻¹ cm⁻¹.

The number of DBCO groups per Fab was then calculated as the ratio of their concentration as measured by UV absorbance.

DBCO-functionalized Fabs were stored at 4° C. if they would be used within a few weeks, and the rest were aliquoted and frozen at −20° C.

Fab conjugation to polymersomes Polymersomes were assembled as described above, with 5% N3-PEG-SS—PPS and 95% mPEG-SS—PPS. DBCO-functionalized Fabs were then reacted with the N3-functionalized polymersomes with Fab-DBCO as the limiting functional group. The smaller volume, the DBCO-functionalized Fab, was added to the tube first, and the larger volume, the N3-functionalized polymersomes, was then added rapidly and immediately mixed by pipetting or vortexing to ensure uniform distribution within the reaction. The click reaction was allowed to proceed overnight at room temperature. The samples were then either purified or transferred to 4° C. until purification.

Fab-functionalized polymersomes were purified by size into PBS either by gravity-driven SEC using Sepharose CL-4B resin or by diafiltration using TFF (MicroKros, 300 kDa MWCO, mPES, 0.5 mm; Repligen) driven either by syringe or, at larger scales, by peristaltic pump (FIG. 20; Fisher Scientific, 13-876-2). The gravity column or TFF flow path was first sterilized using 0.5 M NaOH, then equilibrated with PBS prior to purification, all in a sterile hood.

For purified, Fab-functionalized polymersomes, SAH-MS1-18:polymer mass ratios were typically ˜1:10-1:20, with encapsulation efficiency ˜10-20%. For every formulation, peptide concentrations were measured by LCMS against a AAA-quantified sample, polymer concentrations measured by GPC using refractive index AUC, and Fab concentrations measured using CBQCA against a UV-vis quantified Fab-DBCO control.

Flow cytometry staining Purchased from BioLegend were mouse Fc block (TruStain FcX (anti-mouse CD16/32) antibody, 101320), PE anti-human CD45 (304039, clone HI30), APC anti-human CD19 (363006, clone SJ25C1), and APC-Cy7 anti-human CD20 (302314, clone 2H7). Human Fc block (BD Biosciences 564220, clone 3070) was purchased from Fisher Scientific. Depending on the available lasers on the cytometer used, live/dead (L/D) stain was either a UV-excitation dye (Invitrogen Fixable Blue Dead Cell Stain, L23105) or a violet excitation dye (BioLegend, Zombie Violet Fixable Viability Kit, 423113). To detect mouse-backbone Fabs by flow cytometry, a secondary anti-Fab F(ab′)₂ was purchased from Jackson ImmunoResearch (Alexa Fluor 647 AffiniPure F(ab′)₂ Fragment Donkey Anti-Mouse IgG (H+L), 715-606-151).

For a general staining protocol, cells were washed with PBS and stained with L/D stain 1:500 in PBS for 15 minutes on ice. Fc block was then added directly to the mixture (1:200 for human Fc block, 1:50 for mouse Fc block) for 15 minutes on ice. Antibodies were then added (final dilution 1:100) for 30 minutes on ice. Cells were centrifuged, resuspend in FACS buffer (5% FBS in PBS), and analyzed by flow cytometry.

Cell Culture Human DLBCL cell lines were cultured in RPMI 1640 (Gibco, Thermo Fisher Scientific) supplemented with 10% FBS, 10 mM HEPES (Gibco, 1 M), 2 mM L-glutamine (Gibco, 200 mM), MEM non-essential amino acids (Gibco, 100× solution), and 100 U/mL penicillin-streptomycin (Gibco, 10,000 U/mL) at 37° C. and 5% CO2. Cells were split every 2-3 days. Most cell lines were split to 0.5 million cells per mL, but SU-DHL-5 and OCI-Ly3 were split to 0.1 million cells per mL or lower and not allowed to reach densities higher than 1 million cells per mL. SU-DHL-5 was acquired from ATCC, and OCI-Ly3 and OCI-Ly19 were acquired from DSMZ. The Kline lab kindly shared with us OCI-Ly1, OCI-Ly8, DOHH-2, VAL, and RCK-8.

Cell Death Assays Treatments were prepared in 96-well plates in 50 μL at 2× concentration. Cells were suspended at 0.2 million cells per mL, and 50 μL (10,000 cells) were added to each well and pipette-mixed. The plates were incubated for 24-72 hours, depending on the experiment, then 100 μL of CellTiter-Glo 2.0 (Promega) was added and pipette-mixed, followed by luminescence reading (SpectraMax iD5, Molecular Devices).

Quantitative Real-Time PCR (qRT-PCR) Following relevant drug treatments as indicated, cells were lysed with Trizol (Life Technologies) and total RNA was isolated from each sample using the Direct-zol RNA MiniPrep kit (Zymo Research) per the manufacturer's instructions and quantified (DeNovix DS-11 Spectrophotometer). RNA from each biological replicate (500 ng) was converted to double-stranded cDNA using the Superscript III first strand synthesis reverse transcription kit (Invitrogen) per the manufacturer's directions.

qRT-PCR was performed using TaqMan Master Mix and Gene Expression Probes (Applied Biosystems) for each of the following genes: A1: Hs00187845, B2M: Hs00984230, BAD: Hs00188930, BAK: Hs00832876, BAX: Hs00180269, BCL2: Hs00608023, BCLW: Hs00187848, BCLXL: Hs00236329, BID: Hs00609632, BIM: Hs00708019, BMF: Hs00372937, CDKN1A: Hs00355782, GAPDH: Hs02758991, MCL1: H01050896, NOXA: Hs00560402, PUMA: Hs00248075. Samples were run on the 7500 Fast Real-Time PCR System (Applied Biosciences). Data was analyzed with the ExpressionSuite software utilizing the ΔΔCT method with GAPDH and B2M as two housekeeping genes and DMSO-treated cells as reference samples.

Xenografts Cells were resuspended in either PBS or 50% matrigel in PBS for subcutaneous engraftments using no more than 200 μL. Typically 5 million cells were engrafted per tumor. For disseminated engraftments, no more than 200 μL of cells in PBS were injected through either retro-orbital or tail vein injection.

### Example 1

### PEG-SS—PPS Polymersomes Stability

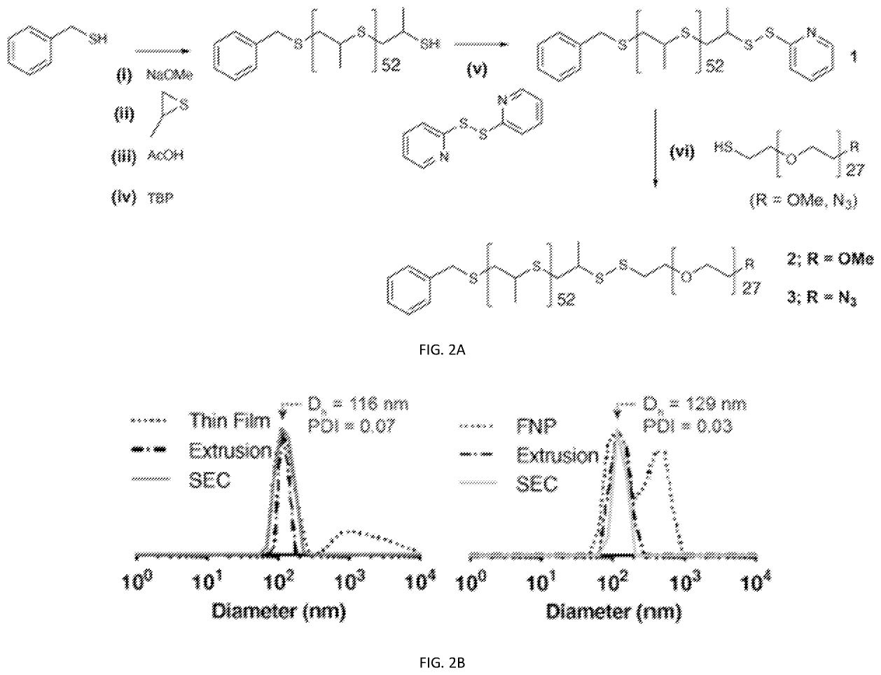

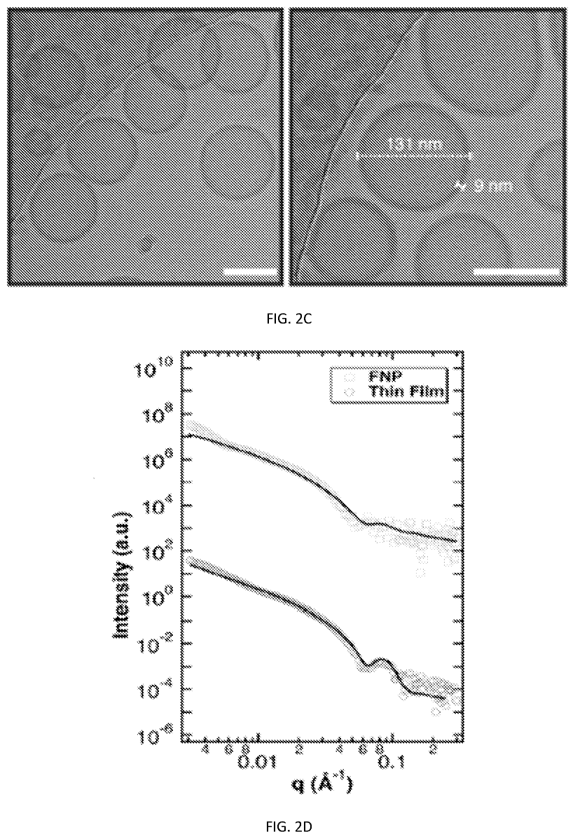

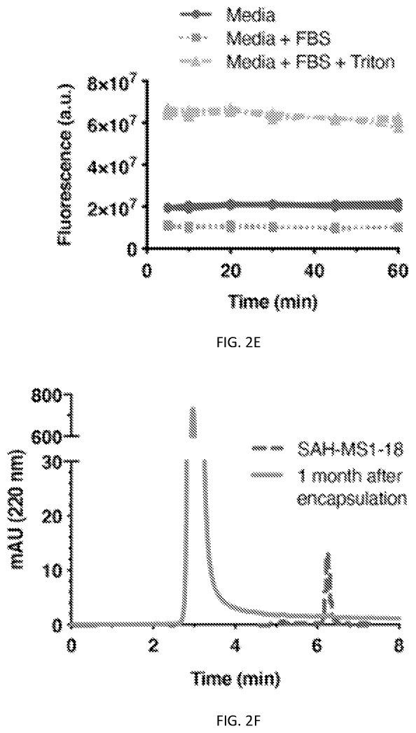

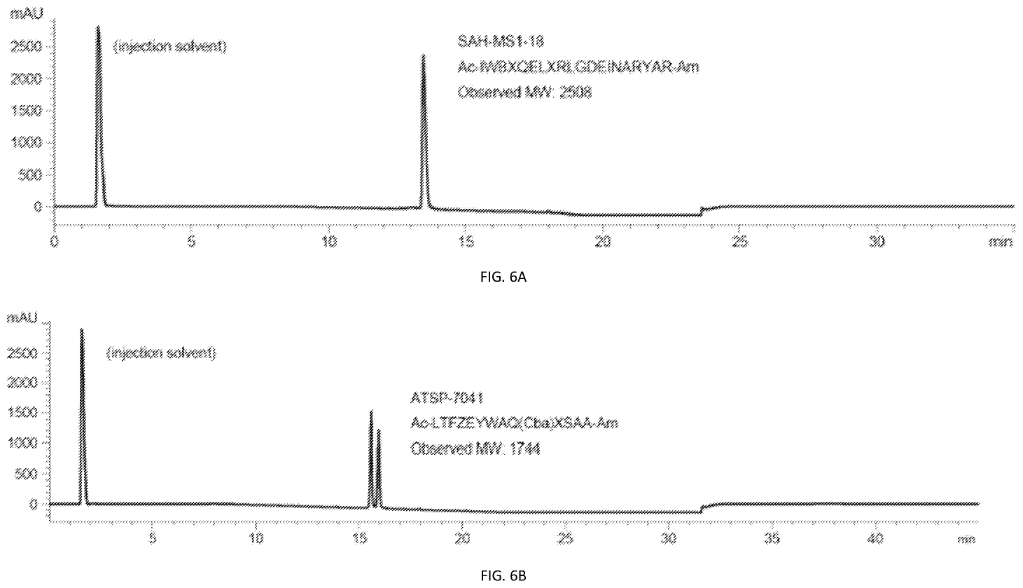

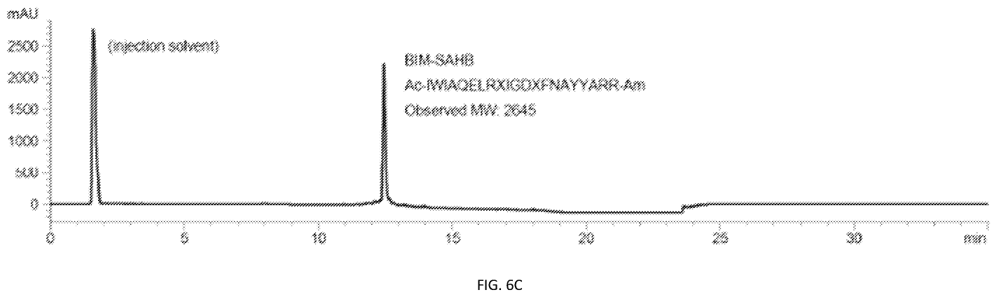

To assemble targeted nanocarriers, the individual components were first synthesized. The PPS homopolymer was synthesized through a living, anionic, ring-opening polymerization (FIGS. 2A and 3). Though some disulfides were present in the polymerization reactions, disulfide exchange proceeded significantly faster than monomer addition, and both unimeric thiol chains (right peak) and dimeric disulfide chains (left peak) underwent a quantitative, living polymerization (FIG. 3A). After polymerization, the disulfide chains were reduced to free thiols (FIG. 3B), capped with a pyridyl disulfide, and purified to generate PPS-PDS (compound 1; FIG. 3C). Thiol-functionalized PEG polymers (mPEG-SH and N₃-PEG-SH) were then reacted with compound 1 to create mPEG-SS—PPS (compound 2; FIG. 4) and N₃-PEG-SS—PPS (compound 3; FIG. 5). Meanwhile, the therapeutic stapled peptide cargoes were synthesized using techniques previously described previously (Refs. 46-47; incorporated by reference in their entireties), confirmed to be >95% pure by LCMS (FIG. 6), and quantified by amino acid analysis (AAA). These components were then all used to assemble polymersomes.

Two previously reported polymersome assembly methods were compared, and both produced indistinguishable polymersomes (FIG. 2B-2D). In the first method, thin-film assembly, the polymer was deposited on the walls of a glass vial in a thin film via evaporation from an organic solvent (DCM). PBS was added to hydrate the film during mixing for several days to gradually form polymersomes. In the second method, ash nanoprecipitation (FNP), a solvent stream (i.e. polymer in THF) and anti-solvent stream (i.e. PBS) were rapidly impinged against each other and diluted into a PBS reservoir to form polymersomes. FNP assembly has previously been reported for this block copolymer as a rapid and scalable way to produce polymersomes (Refs. 48-49; incorporated by reference in their entireties). A confined impingement jets with dilution (CIJ-D) device was made using a design and dimensions published previously (Ref. 50; incorporated by reference in its entirety), except instead of drilling channels out of a solid block of material, a computer aided design (CAD) file was used to 3D print the device with patent channels (FIG. 7). Both thin-film- and FNP-assembly produced a primary population of polymersomes approximately 120 nm in diameter, but larger aggregates were also present in each case (FIG. 2B). All samples were therefore extruded through a 100 nm pore-size membrane to create monodisperse polymersomes and then purified by either SEC or tangential-flow filtration (TFF) diafiltration. Cryo-EM was used to visually confirm that the assemblies were indeed polymersomes, as opposed to other structures that have been reported from these block copolymers at other block ratios (FIG. 2C) (Refs. 48 and 51; incorporated by reference in their entireties). To further confirm their vesicular structure at an ensemble level, Small Angle X-ray Scattering (SAXS) data were fitted using a spherical vesicle model, and the nanoparticles from both assembly methods were well-represented as spherical, hollow vesicles with diameter and bilayer thickness corresponding to those seen in cryo-EM (FIG. 2D). Of the two assembly methods, the FNP method was more easily scalable and allowed for rapid encapsulation of therapeutic cargoes.

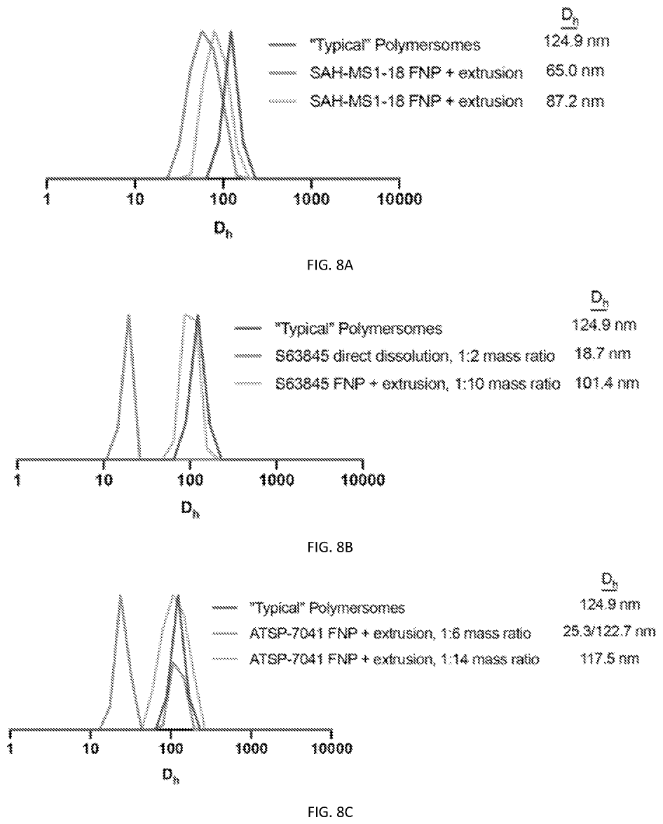

While most polymersome formulations made yielded polymersome formulations of about the same size (i.e. 120-130 nm), some drug encapsulations have produced other structures (FIG. 8) presumably micelles in some cases and smaller polymersomes in other cases. SAH-MS1-18, when encapsulated in polymersomes using the FNP method, consistently yielded slightly smaller polymersomes with hydrodynamic diameters of 65-90 nm before extrusion (FIG. 8A). When S63845 or ATSP-7041 were encapsulated with high drug concentrations, much smaller structures formed (FIGS. 8B and 8C), and for the S63845 sample, cryo-EM confirmed these were micelles. For both drugs, when the amount of drug relative to polymer was decreased, more typical polymersomes were formed. Of note, using the inverse direct dissolution method previously described (Ref. 52; incorporated by reference in its entirety), S63845 and ATSP-7041 were both highly soluble in a pipettable polymer melt made from mixing PEG-SS—PPS and PEG(500)DME, presumably these drugs have favorable interactions with the PEG-SS—PPS block copolymer during drug encapsulations. It seems that drugs that favorably interact with PEG-PPS, including two hydrocarbon stapled peptides, can influence the structure of polymersomes formed in the presence of very high concentrations of drug relative to polymer.

The stability of PEG-SS—PPS polymersomes was tested in the presence of fetal bovine serum (FBS; FIG. 2E). Polymersomes encapsulating a hydrophilic dye, calcein, at self-quenching concentrations, were used to detect polymersome disruption via fluorescence dequenching. When the polymersome stock solution was diluted into cell culture media and incubated at 37° C. for 1 hour, there was no detectable polymersome disruption (FIG. 2E, Media). When the polymersomes were diluted into media with 10% FBS, the fluorescence still remained constant, indicating no polymersome disruption due to serum proteins (FIG. 2E, Media+FBS). As a positive control, a detergent (Triton X-100) was added to completely disrupt the polymersomes and release calcein, and this caused a large increase in fluorescence intensity (FIG. 2E, Media+FBS+Triton). PEG-SS—PPS polymersomes were highly stable in the presence of serum proteins, in agreement with the stability generally associated with polymersomes as a class of nanoparticles.

Polymersomes encapsulating SAH-MS1-18 were stored for 1 month at 4° C. in PBS, then the sample was analyzed by aqueous SEC HPLC to detect any peptide released (FIG. 2F). No detectable amount of free peptide had leaked out of the polymersomes during 1 month of storage, highlighting the stability of stapled peptide encapsulation and compatibility with long-term storage in PBS at 4° C.

Notably, polymersome encapsulation of SAH-MS1-18 also greatly enhanced the aqueous solubility of the peptide, which is a crucial consideration for intravenous injection of sufficient doses. On larger scales, PSOM_{SAH-MS1-18} was concentrated by TFF such that the average SAH-MS1-18 concentration in the solution was in the millimolar (mM) range (e.g. 2.7 mM in the overall solution, but all locally concentrated inside polymersomes), and no aggregation was observed by eye or DLS. This was more than 10 times the solubility limit of the peptide alone in PBS.

### Example 2

αCD19 Polymersomes Deliver Cargo into DLBCL Cells Specifically Via CD19

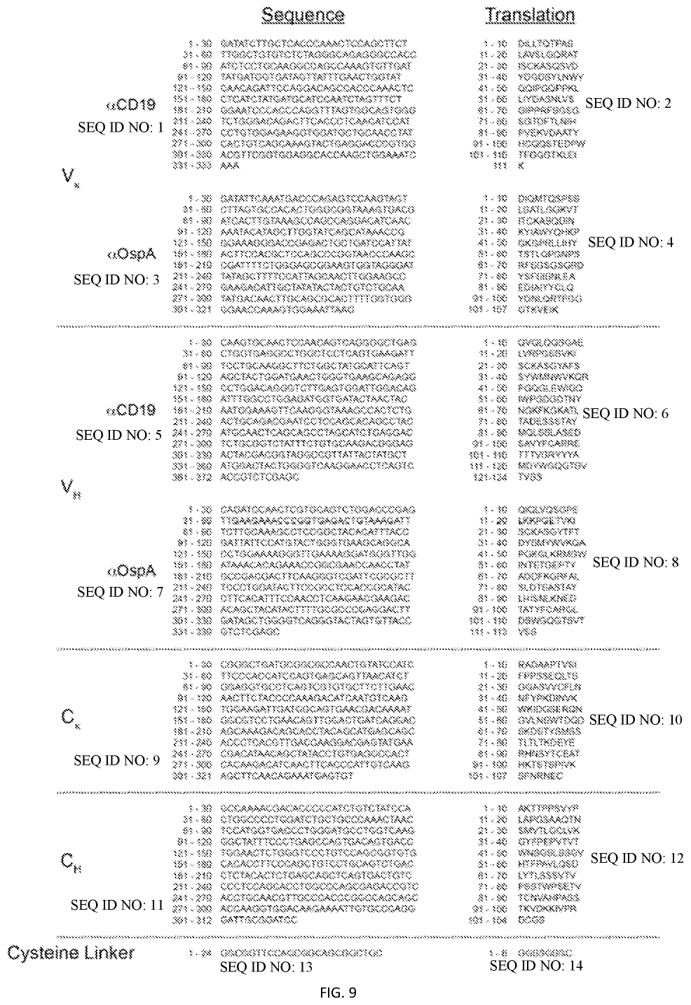

A Fab specific for human CD19 (αCD19) was designed with an added cysteine linker (αCD19-cys) for site-specific conjugation to polymersomes. The variable regions of the αCD19-cys Fab were designed from the HD37 mouse-anti-human-CD19 IgG, with constant regions from mouse IgG consensus sequences (FIG. 9). The cysteine linker was added at the C-terminus of the heavy chain, opposite the antigen-binding face, with a short, flexible, hydrophilic spacer and a terminal cysteine (FIG. 10A). To generate non-binding control Fabs, the variable regions were grafted from a published sequence targeting the xenoantigen Outer surface protein A (OspA) of Borrelia burgdorferi, while the constant regions remained unchanged (FIG. 9). The four Fabs (αCD19, αCD19-cys, αOspA, and αOspA-cys) were cloned in DH5α, expressed in HEK293T cells, and purified by Protein G affinity chromatography (FIG. 10B). The antigen-specific binding of αCD19-cys to CD19+DLBCL cells was tested, and it bound specifically, with no apparent influence from the encoded cysteine linker (FIG. 10C).

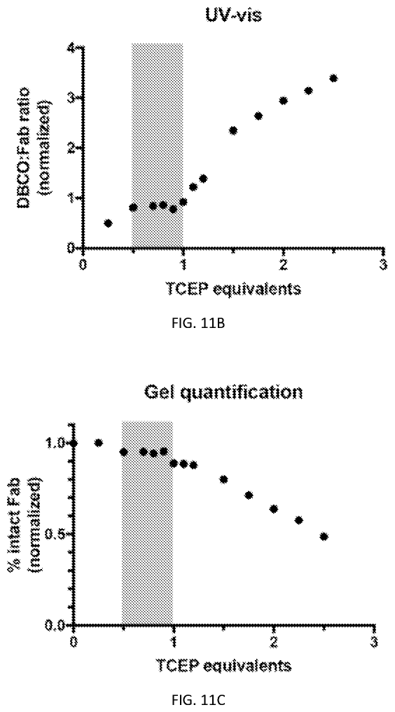

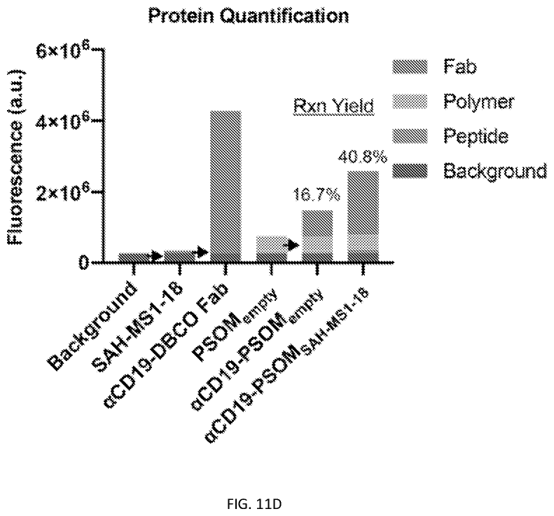

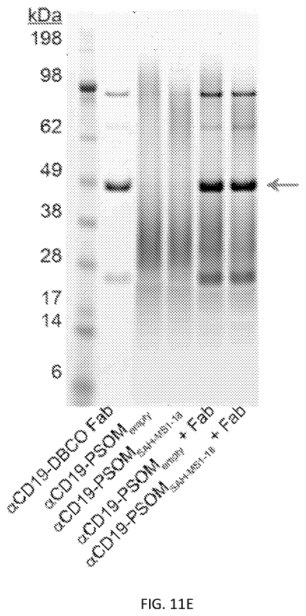

The Fabs' cysteine linker was functionalized with a DBCO handle for click-chemistry attachment to the surface of the polymersomes (FIG. 11A). When the Fabs were initially purified, the thiol on the cysteine linker was unreactive. Solvent-accessible cysteines on recombinant proteins secreted from mammalian cells may be initially disulfide-bonded with small molecule thiols, such as cysteine and glutathione (Ref. 58; incorporated by reference in its entirety). By titrating the amount of reducing agent, TCEP, the solvent-accessible cysteine linker was specifically reduced and converted to a DBCO handle without disrupting internal disulfides (FIGS. 11B and 11C).

The amount of TCEP that reduced only the terminal thiol was a range of values, rather than a single point. The range from 0.5-1 equivalents of TCEP was a stable range to reduce precisely 1 equivalent of reactive thiol on the Fabs (FIGS. 11B and 11C). This range may be explained by the relative reducing potentials of the thiols in the system. One TCEP molecule will generate one Fab-thiol and one small molecule thiol, and that liberated small molecule thiol, presumably cysteine or glutathione, appears to favorably reduce the terminal thiol on a second Fab. Therefore, 0.5 equivalents of TCEP generated 1 equivalent of Fab-thiol and, 0.5 equivalents of a small molecule disulfide. The next 0.5 equivalents of TCEP (0.5-1 equivalents total) are then presumably consumed in reducing the small molecule disulfides and don't further reduce internal disulfides in the Fab. This window, then, from 0.5-1 equivalents of TCEP per Fab, was a safe range to precisely functionalize the Fabs with a DBCO click chemistry handle and reliably produced DBCO:Fab ratios of 1. These are equivalents with respect to unimeric (not disulfide dimeric) intact (not free heavy or light chain) Fab, as determined by UV absorbance at 280 nm for total protein concentration combined with Coomassie-stained SDS-PAGE gel quantification for the relative percentage of each species. TCEP was chosen as the reducing agent due to its powerful reducing potential nearly independent of pH and its relative nonreactivity with maleimides, which allows the reduced-Fab TCEP mixture to be directly reacted with the maleimide-DBCO linker without any workup and chance for re-oxidation.

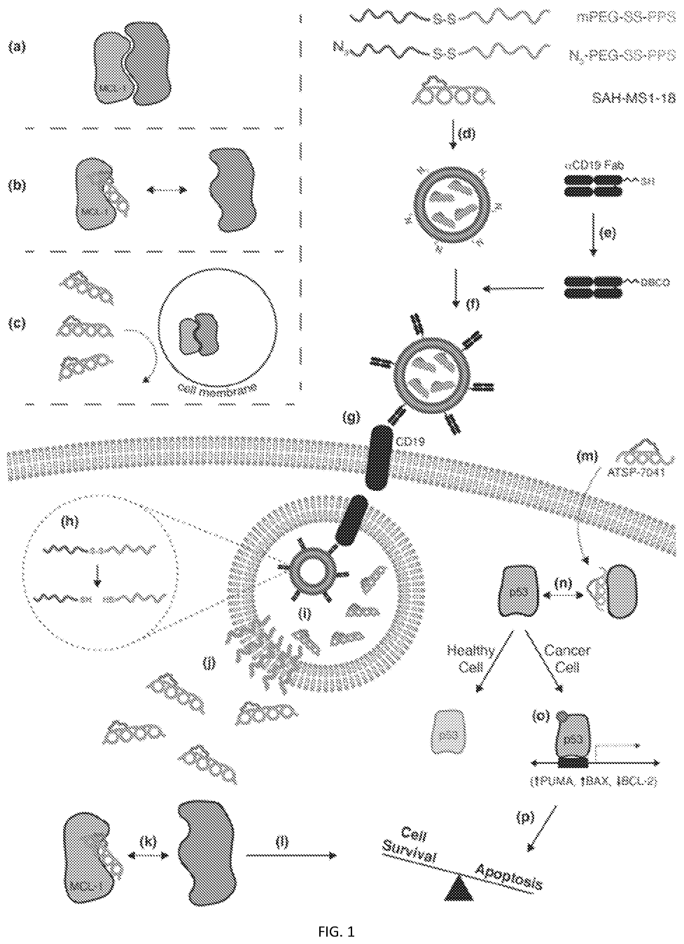

The DBCO-functionalized Fabs were then “clicked” onto the polymersomes. Polymersomes were generated with a range of Fab densities on the surface by using the N₃ on the polymersome surface as the excess functional group (5% N₃-PEG-SS—PPS, 95% mPEG-SS—PPS) and adding different molar amounts of Fab-DBCO into aliquots from a common polymersome stock solution (FIG. 1C). Using reaction stoichiometries targeting 0.1%, 0.5%, and 1% polymer functionalization, low (+), medium (++), and high (+++) Fab densities were generated. The resulting Fab-polymersomes were purified by size to remove any non-conjugated Fab. The amount of Fab attached to the polymersome surface could be quantified using the CBQCA protein quantification assay according to the manufacturer's instructions (representative example in FIG. 11D), and the successful removal of non-conjugated Fab could be verified using a Coomassie-stained SDS-PAGE gel (FIG. 11E). The aggregation number could be precisely measured using light scattering experiments, but the available data was used for estimation.

First, using the density of PPS and volume of the PPS layer of the polymersome, how many chains there are per particle could be estimated assuming the PPS layer has a density equivalent to bulk PPS. This is likely an upper-limit estimation of the number of chains per particle. From the large scale synthesis of PPS-PDS, the density of the pure bulk homopolymer was 1.169 g/mL. For a polymersome with roughly 130 nm diameter and a 9 nm PPS layer thickness from cryo-EM, the volume of the PPS layer can be roughly estimated as

$$Volume = \frac{4}{3} {π ( r nm )}^{3} - \frac{4}{3} {π ( r - \frac{9}{2} nm )}^{3} with r = \frac{130}{2} = 65 nm , \\ or Volume = 222 , 759 {nm}^{3} .$$

Then with the volume of the PPS layer (222,759 nm³), the density of bulk PPS (1.169 g/mL), and the average molar mass of PPS₅₃ (3181 g/mol), the number of chains per particle can be estimated as Volume×Density÷Molar Mass=49; 281 polymers per particle.

A simple Nanoparticle Tracking Analysis (NTA) measurement measured the concentration of nanoparticles (nanoparticles/mL) for a sample with a known concentration of polymer (mg/mL) with a known molar mass (5,324 g/mol for mPEG28-SS—PPS₅₃). From this, 15,632 polymers per particle were estimated. While neither of these methods are as accurate as measuring aggregation number by light scattering, both gave a rough estimation that was on the order of magnitude of 15,632-49,281 polymers per polymersome for a 130 nm Dh polymersome made of PEG₂₈-SS—PPS₅₃. Functionalizing 1% of the polymers in the outer bilayer (assuming half, and no flipping of the N₃ groups across the bilayer) means adding roughly 78-246 Fabs per particle, and functionalizing 0.1% would mean roughly 8-25 Fabs per particle, assuming 100% reaction efficiency. Reaction efficiencies were typically 10-40% and seemed to vary based on the concentration of the samples during the reaction.

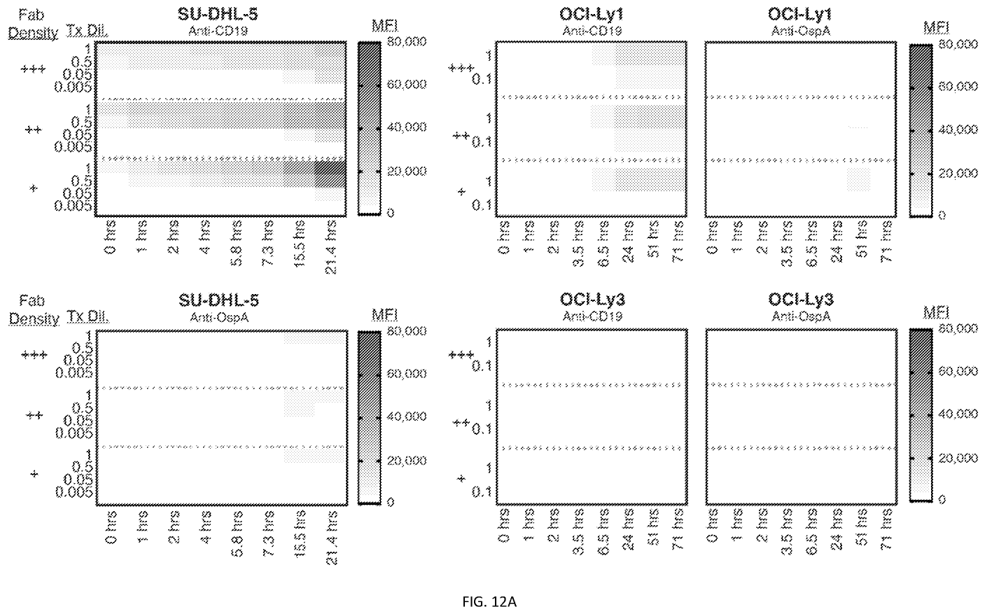

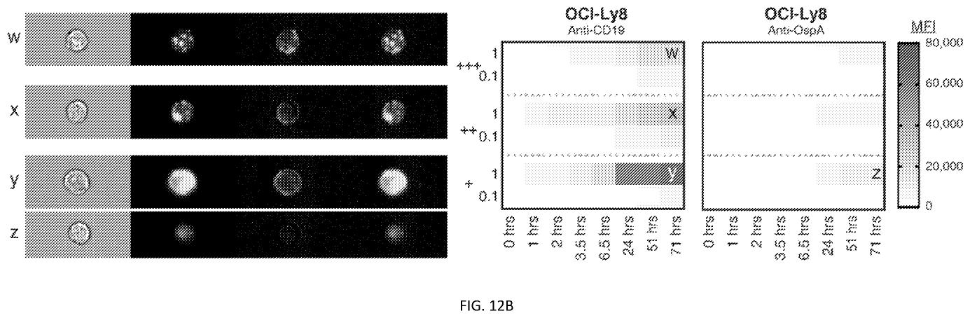

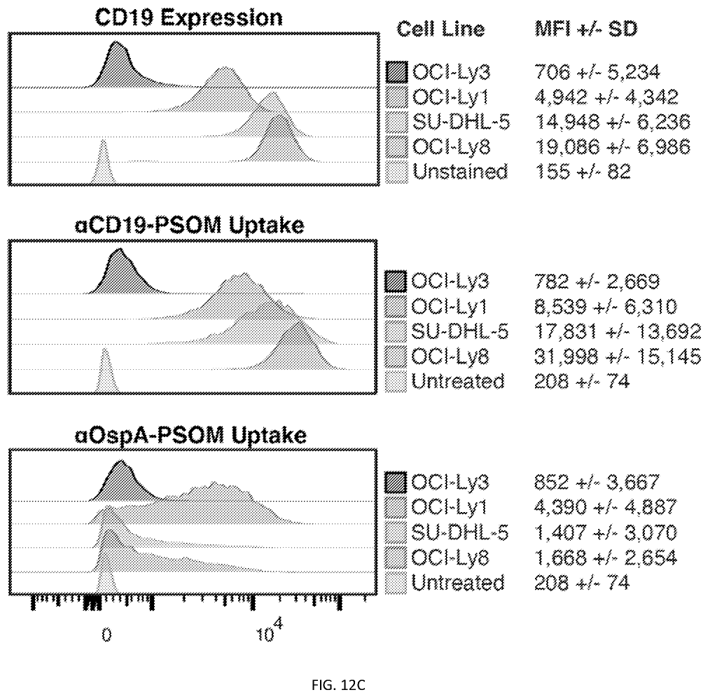

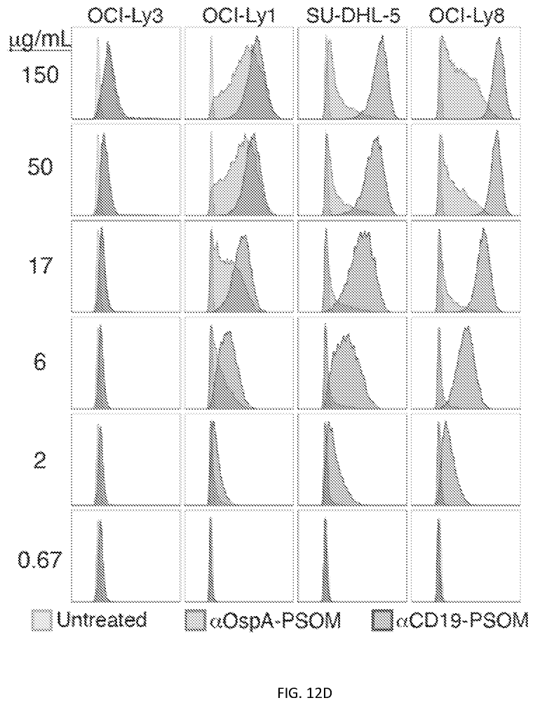

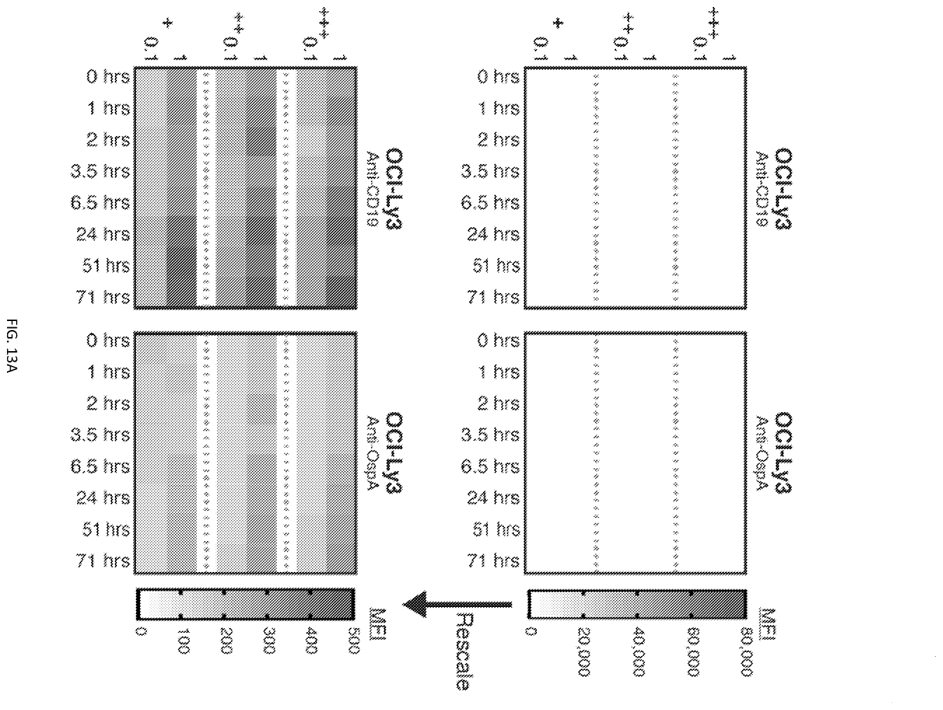

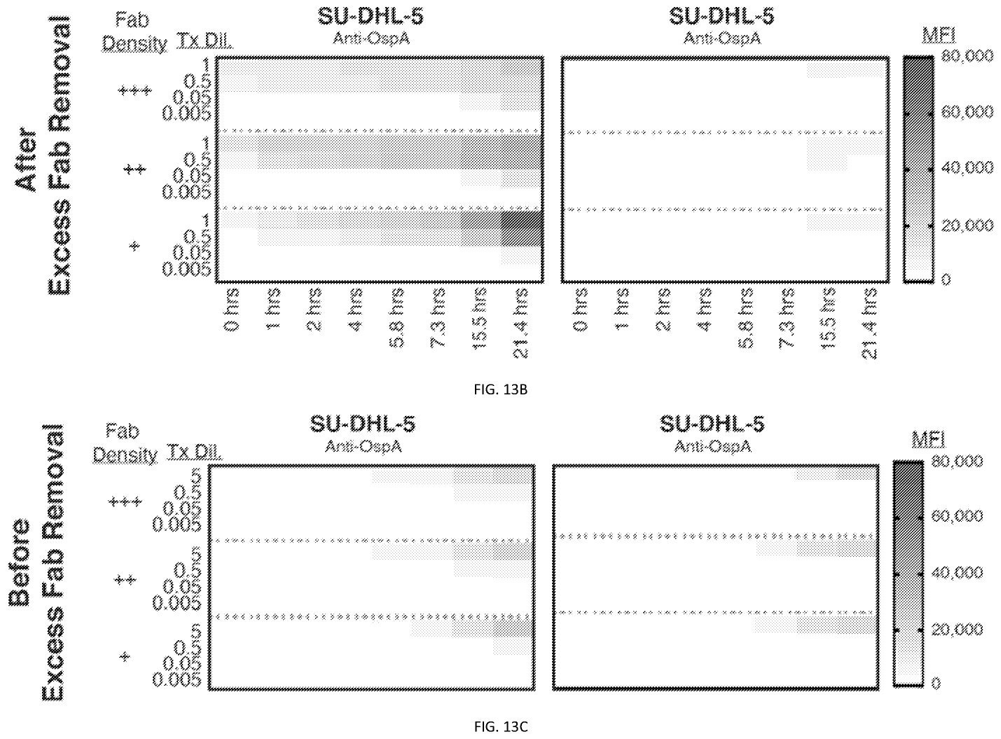

To measure uptake, a self-quenching solution of the hydrophilic fluorophore calcein was encapsulated into polymersomes and attached either αCD19 Fab (αCD19-PSOM_{calcein}) or an irrelevant Fab (αOspA-PSOM_{calcein}) to the surface at varying densities (high (+++), medium (++), and low (+)). Four DLBCL cell lines (SU-DHL-5, OCI-Ly1, OCI-Ly3, and OCI-Ly8) were treated with the fluorescence-quenched polymersomes and measured uptake by flow cytometry. In each cell line, antigen-specific, dose-dependent, and time-dependent accumulation of calcein fluorescence was observed (FIG. 12A). Even OCI-Ly3, which expresses low but non-zero levels of CD19 (FIG. 12), exhibited low levels of antigen-specific uptake (FIGS. 13A and 13B). Regardless of cell line, the αCD19-PSOM_{calcein} with the lowest Fab densities (+) were endocytosed to the greatest degree. As further evidence of active targeting, if the same treatments were performed without the final purification step to remove non-conjugated Fabs from the samples, antigen-specific uptake was almost completely blocked by the contaminating free Fabs (FIGS. 13B and 13C).

To confirm that the polymersomes were enhancing intracellular calcein accumulation and dequenching rather than simply binding more to the cell surface, the same samples were imaged using ImageStream imaging cytometry (FIG. 12B). The lower Fab densities enhanced antigen-specific uptake and diffuse, intracellular calcein (green) accumulation. Extracellular polymersomes on the cell surface were stained using a fluorescent anti-Fab antibody, and the extracellular anti-Fab staining (magenta) did not overlap with the intracellular calcein staining (green), confirming that the diffuse calcein signal was a result of enhanced intracellular accumulation and fluorescence dequenching rather than simply increased extracellular binding.

The uptake of αCD19-PSOM_{calcein} was also highly antigen specific. Uptake of αCD19-PSOM_{calcein} in each cell line correlated with expression levels of CD19, while uptake of αOspA-PSOM_{calcein} was less, more heterogeneous, and uncorrelated with CD19 expression (FIG. 12C). This trend was consistent across a range of doses (FIG. 12D).

αCD19-PSOMs are endocytosed antigen-specifically with lower Fab densities causing the greatest intracellular accumulation. This Fab density formulation (+) was used for further experiments with therapeutic cargoes.

### Example 3

### Polysome-Mediated Intracellular Delivery Enhances the Therapeutic Efficacy of BH3-Mimetic Stapled Peptides

Calcein was a useful model cargo to optimize polymersome uptake into DLBCL cells, and next polymersomes were made encapsulating the therapeutic cargo, SAH-MS1-18 (Ref. 23; incorporated by reference in its entirety), to ultimately test the polymersomes' ability to improve the intracellular delivery and efficacy of stapled peptides.

After encapsulating SAH-MS1-18 in polymersomes (PSOM_{SAH-MS1-18}) and functionalizing them with Fabs (αCD19-PSOM_{SAH-MS1-18} and αOspA-PSOM_{SAH-MS1-18}), the ability of SAH-MS1-18 to induce apoptosis in DLBCL were tested when it was either used as a free peptide or when its intracellular delivery was facilitated by PEG-SS—PPS polymersomes.

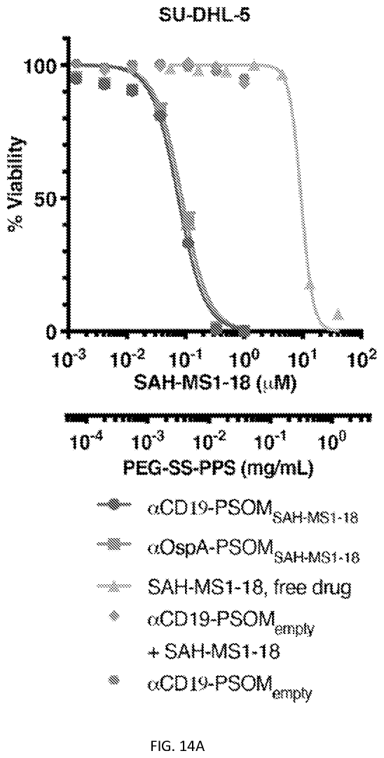

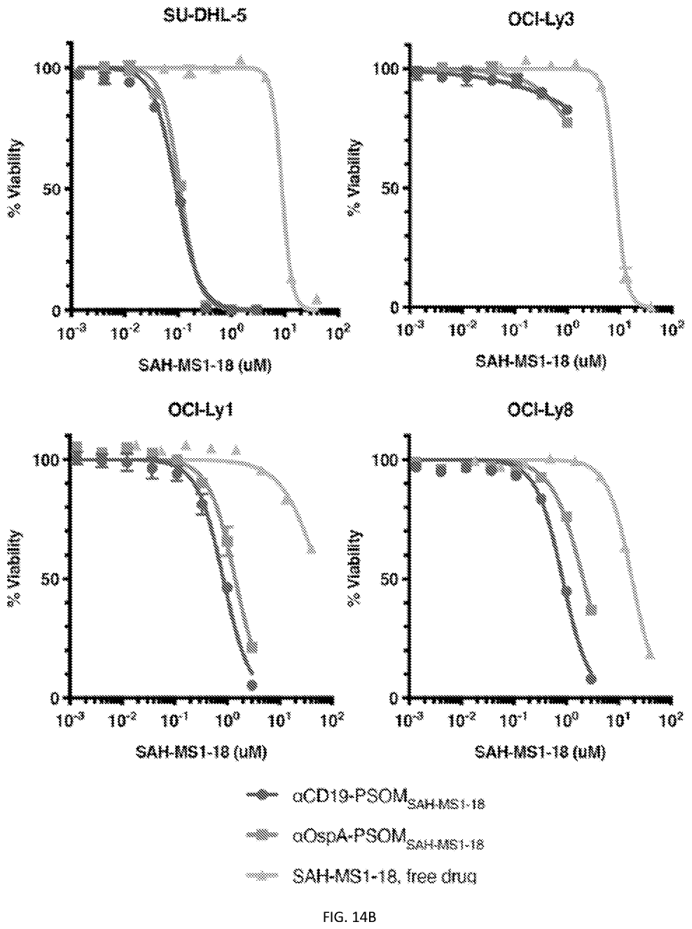

First, SU-DHL-5 was treated with equivalent doses of SAH-MS1-18 either as a free drug, inside of αCD19- or αOspA-PSOMs, or on the outside of empty αCD19- or αOspA-PSOMs (FIG. 14A). Delivery of SAH-MS1-18 inside of polymersomes enhanced its potency by ˜100-fold. Importantly, when the same doses of peptide were used but on the outside of empty polymersomes, cell death was completely eliminated. This confirmed that the greatly enhanced potency was due to the facilitated delivery, rather than any non-specific toxicity due to the combination of materials. Other DLBCL cell lines, including OCILy1, OCI-Ly3, and OCI-Ly8, were treated (FIG. 14B). Delivery inside of polymersomes enhanced the potency of SAH-MS1-18 by ˜10-fold in OCI-Ly1 and OCI-Ly8. OCI-Ly3, which endocytosed very low levels of αCD19- or αOspA-PSOMs (FIGS. 12 and 13A-13B) exhibited little cell death. Even for this qualitatively cell permeable stapled peptide, intracellular delivery using PEG-SS—PPS polymersomes greatly enhanced its efficacy.

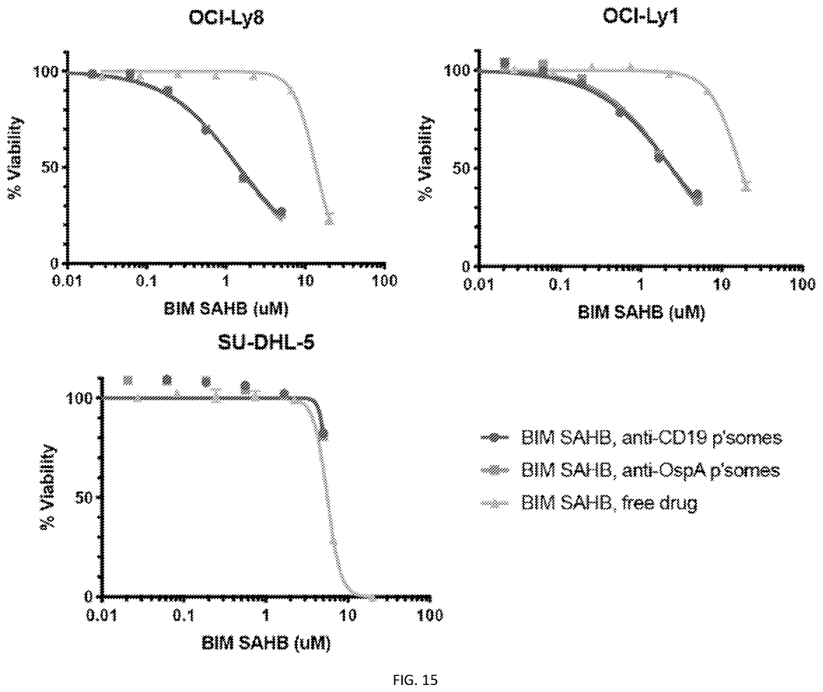

To confirm this delivery benefit was not unique to SAH-MS1-18, another apoptosis-inducing stapled peptide, BIM SAHB (Refs. 21, 60-61; incorporated by reference in their entireties), was delivered into DLBCL cells using polymersomes (FIG. 15). The potency of BIM SAHB was improved 10× by polymersome delivery into OCI-Ly1 and OCI-Ly8. Importantly, SU-DHL-5 was not sensitive to BIM SAHB delivered in polymersomes, even though it was extremely sensitive to SAH-MS1-18 delivered in the same way. This highlights the mechanistic specificity of these peptides' induction of apoptosis and the benefit this system provides by enhancing cellular uptake.

Unexpectedly, αOspA-PSOMs loaded with therapeutic cargoes were almost as potent as αCD19-PSOMs (FIGS. 14 and 14), even though αCD19-PSOMs facilitated greater uptake of a calcein model cargo (FIG. 12). One possible explanation for this could be the threshold character of apoptosis as opposed to the continuous scale of calcein fluorescence. If a small amount of peptide is delivered non-specifically into the cell by αOspA-PSOMs, and if it is enough to induce apoptosis, then no further accumulation could be facilitated by CD19 targeting and appreciated in a cell death assay. These data suggest that the majority of improved potency in vitro is likely due to facilitated endosomal escape and/or protecting the peptide cargo from serum protein sequestration. For targeted nanocarriers, the benefits of targeting are usually more pronounced in vivo than in vitro.

### Example 4

### P53-Reactivation Primes DLBCL for Cell Death by MCL-1 Inhibition and Sensitizes DLBCL to αCD19-PSOM_{SAH-MS1-18}

Tumor suppressor protein p53 is known to modulate transcription of a number of BCL-2 family members in a pro-apoptotic way. A p53-reactivating stapled peptide, ALRN-6924, is currently in clinical trials. While the sequence of ALRN-6924 is proprietary and unpublished, its pre-clinical predecessor, ATSP-7041, has a published sequence and has used by multiple groups for p53 reactivation. ATSP-7041 has been highly optimized to be cell-permeable and drug-like, and its therapeutic efficacy is not negated by serum proteins (Ref. 22; incorporated by reference in its entirety). ATSP-7041 is also one of the few stapled peptides that has been successfully applied in vivo. ATSP-7041 was used as a p53-reactivating stapled peptide to prime DLBCL for apoptosis.

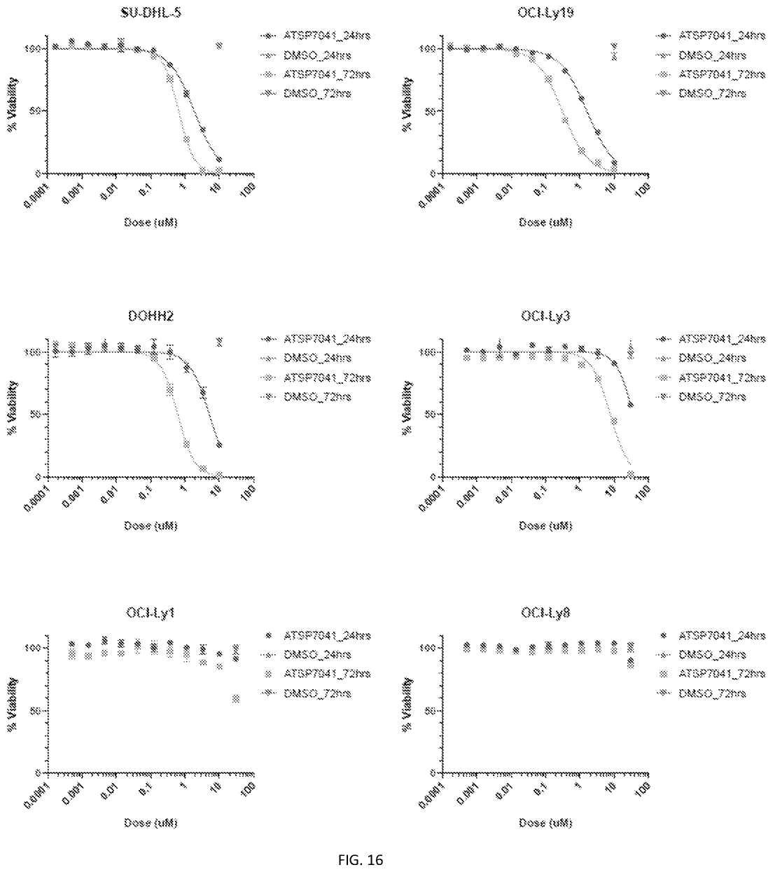

Interestingly and surprisingly, p53 primed DLBCL cell lines for cell death specifically with increased sensitivity to MCL-1 inhibition rather than to other anti-apoptotic proteins such as BCL-2 or BCL-XL. To determine therapeutically-relevant treatment concentrations, the cell death sensitivity of DLBCL cell lines to ATSP-7041 at 24 and 72 hours (FIG. 16) was tested. For three cell lines with wildtype p53 (SU-DHL-5, OCI-Ly19, and DOHH2), 1 μM ATSP-7041 was an amount that induced some apoptosis at 24 hours and a lot more apoptosis by 72 hours. A fourth DLBCL cell line with wildtype p53, OCI-Ly3, was less sensitive to ATSP-7041 treatment, and this cell line was included as a more resistant WTp53 control. Two DLBCL cell lines with mutant p53 (OCI-Ly1 and OCI-Ly8) had no cell death in response to ATSP-7041, except a small amount at the highest dose, 30 μM, for 72 hours of treatment. This 30 μM dose is higher than the highest dose found in the literature for in vitro treatments (10 μM; Ref. 22; incorporated by reference in its entirety), and this cell death is likely non-specific due to the high dose. After 24 hours of treatment, ATSP-7041 has previously been shown to induce p53 transcriptional activation (Ref. 22; incorporated by reference in its entirety), and with these data, 1 μM was chosen as the 24-hour treatment dose to evaluate BCL-2 family changes in response to p53 re-activation.

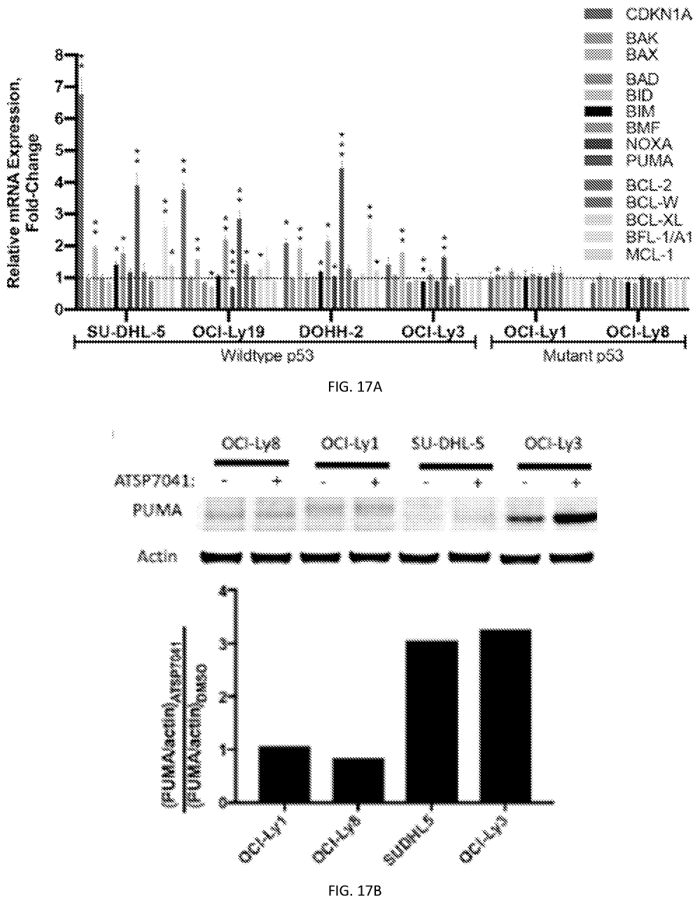

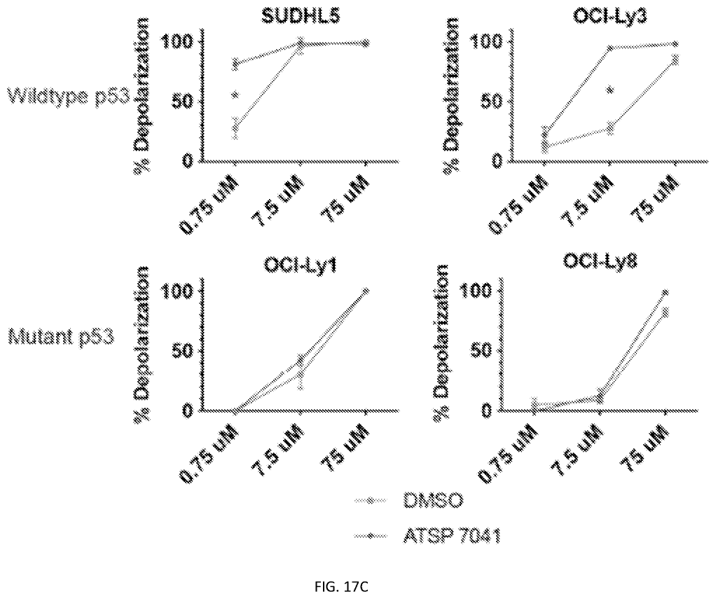

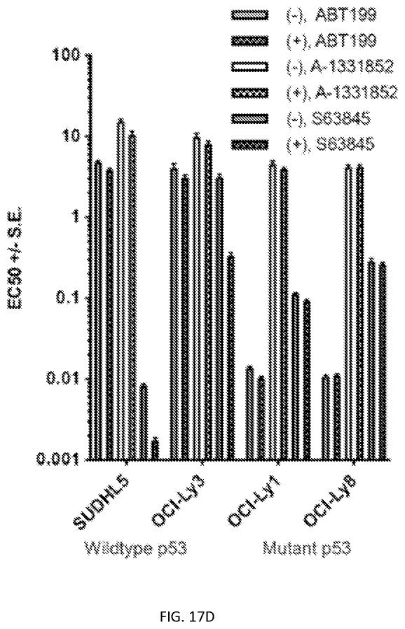

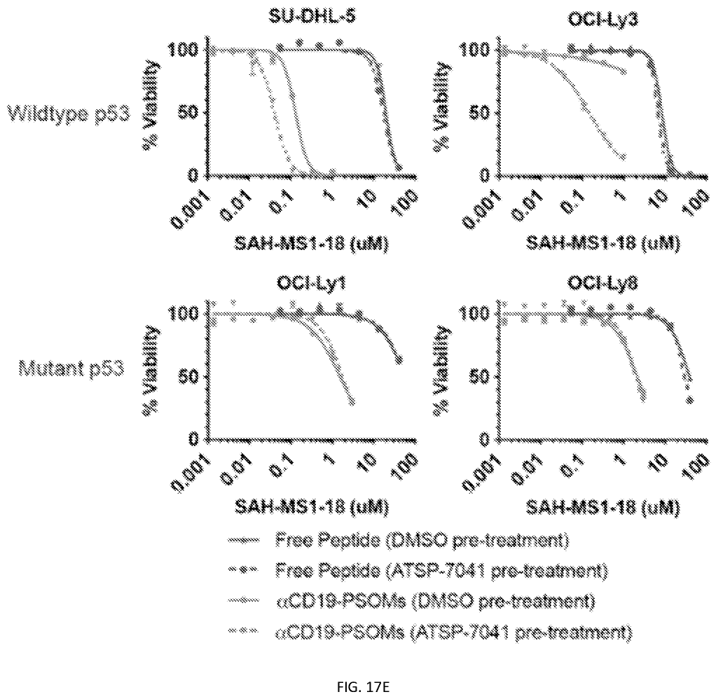

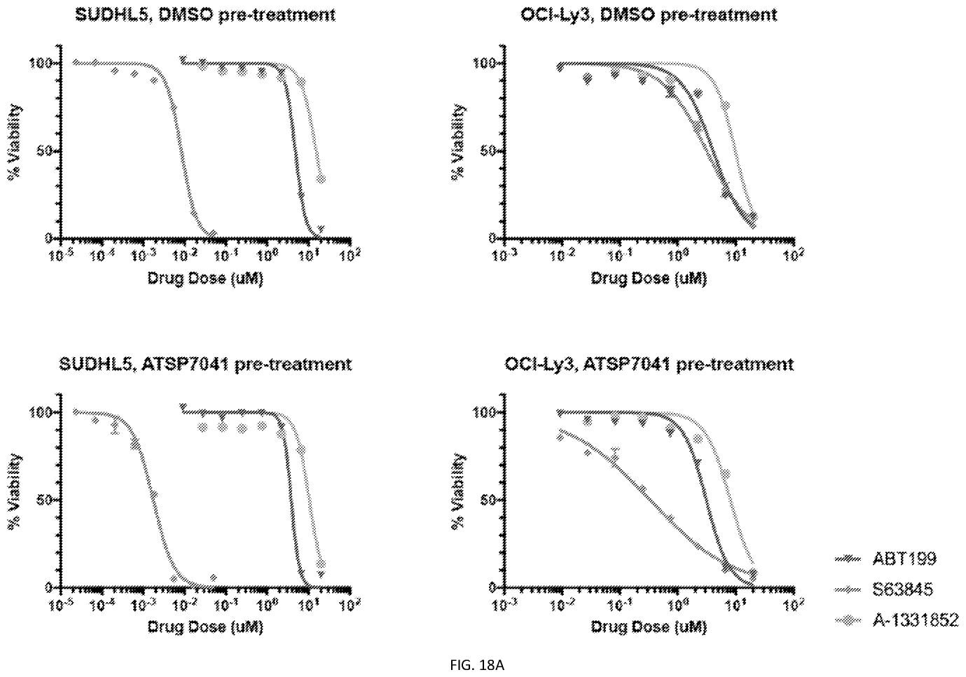

When DLBCL cell lines were treated with the p53-reactivating stapled peptide ATSP-7041 for 24 hours, DLBCL with WTp53 exhibited transcriptional changes within the BCL-2 family consistent with known p53 transcriptional targets (FIG. 17). First, CDKN1A (p21) transcription was highly upregulated, indicating robust p53-reactivation. Within the BCL-2 family, the mRNA of p53 upregulated modulator of apoptosis (PUMA) was strongly upregulated across each of the cell lines with WTp53. BAX, an effector of apoptosis and another known p53-transcriptional target, was also upregulated across each of the WTp53 cell lines. Interestingly, NOXA mRNA appeared unchanged after p53-reactivation. NOXA is a canonical transcriptional target of p53, though it is also regulated by multiple other transcription factors. In general, the WTp53 cell lines responded to p53 reactivation by increasing expression of PUMA and BAX, transcriptional changes consistent with priming the cells for apoptosis.

These transcriptional changes after p53-reactivation were monitored for the effect on protein levels of PUMA (FIG. 17B). Consistent with the changes in PUMA mRNA, PUMA protein was also upregulated after p53-reactivation in DLBCL lines with wildtype p53 (i.e. SU-DHL-5, OCI-Ly3) but not in lines with mutant p53 (i.e. OCI-Ly1, OCI-Ly8).

Mitochondrial outer membrane permeabilization (MOMP) by BAX and BAK, and the resulting mitochondrial depolarization, is the point-of-no-return when a cell initiates the feed-forward process of apoptosis. Cells' sensitivities to mitochondrial depolarization and apoptosis can be measured by permeabilizing the cell membrane and treating with varying concentrations of a BIM BH3 peptide, the BH3 binding domain of pan-activating protein BIM. The more “primed to die” the cells are, the less BIM BH3 peptide is required to induce mitochondrial depolarization. After treatment with p53-reactivator ATSP-7041, cell lines with wildtype TP53 (i.e. SU-DHL-5, OCI-Ly3) were significantly more “primed to die” than vehicle-treated controls (FIG. 17C). In agreement with the mRNA and protein changes in the BCL-2 family, p53-reactivation functionally sensitized DLBCL to MOMP.

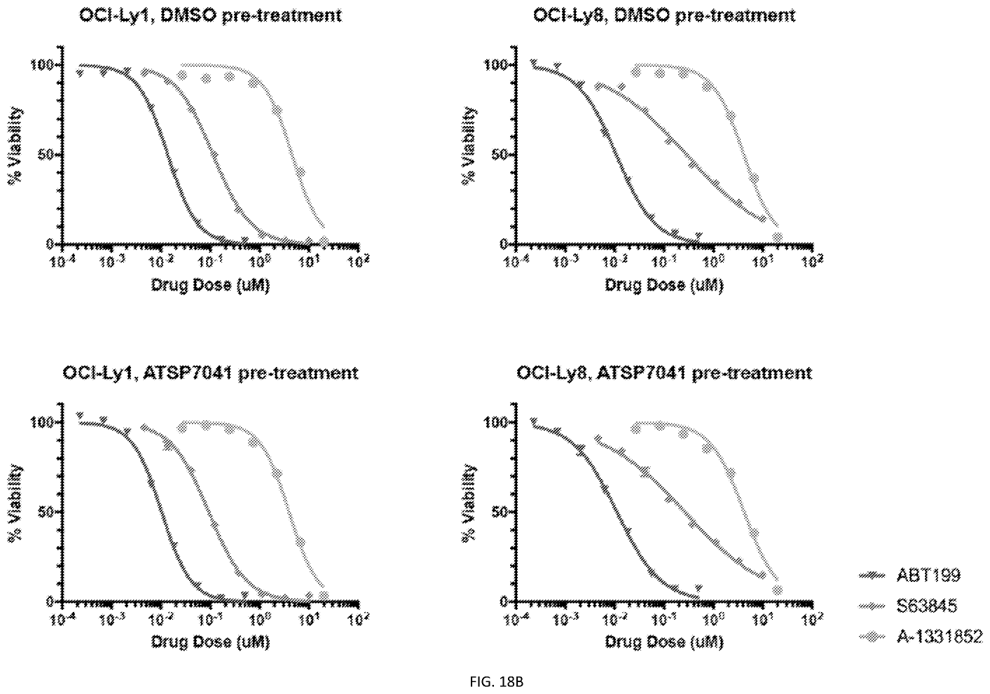

While DLBCL was primed for apoptosis after p53-reactivation, it was not yet clear which anti-apoptotic proteins the surviving cells were relying on for survival, because increased levels of PUMA and BAX could theoretically be sequestered by any of the anti-apoptotic proteins in the BCL-2 family. Three of the anti-apoptotic proteins, BCL-2, BCL-XL, and MCL-1, are the most commonly implicated in chemoresistant and chemorefractory cancers, and each of these three proteins has recently been successfully inhibited using specific small molecule therapeutics. Whether or not p53-reactivation changed the sensitivity of these DLBCL cell lines to therapeutic inhibition of BCL-2 by ABT-199 (a.k.a. venetoclax), BCL-XL by A-1331852, or MCL-1 by S63845 was tested (FIGS. 17D and 18). Cells were pre-treated with ATSP-7041 (+) or a vehicle control (−) for 24 hours, washed, and then treated with varying doses of each therapeutic, and dose-death curves were fitted to calculate EC₅₀ sensitivities. After p53-reactivation, the cell lines with WTp53 were much more sensitive to the MCL-1 inhibitor (S63845). Both an MCL-1-sensitive cell line (SU-DHL-5) and a relatively resistant cell line (OCI-Ly3) were made much more sensitive to MCL-1 inhibition by first reactivating p53 with ATSP-7041. Surprisingly, there was no notable change in sensitivity to the BCL-2 inhibitor (ABT-199) or BCL-XL inhibitor (A-1331852), even though each of these anti-apoptotic proteins was capable of sequestering PUMA and BAX. This highlights MCL-1 as a synergistic therapeutic target with p53-reactivation, such as by ATSP-7041.

The sensitivity of DLBCL to the stapled peptide MCL-1 inhibitor, SAHMS1-18, delivered either in polymersomes or as free drug, with and without p53-reactivation was tested (FIG. 17E). After priming cells for 24 hours with ATSP-7041 and washing off the drug, each cell line was then treated for 72 hours with equivalent doses of SAH-MS1-18, either in polymersomes or as free peptide. DLBCL with WTp53 was made more sensitive to SAH-MS1-18 delivered as αCD19-PSOM_{SAH-MS1-18} after p53-reactivation. As with the small molecule inhibitor of MCL-1 (S63845), a sensitive cell line (SU-DHL-5) was made even more sensitive by p53-reactivation. Notably, a resistant cell line, OCI-Ly3, became a sensitive cell line simply by reactivating p53. OCI-Ly3 also endocytosed very small amounts of αCD19-PSOMcalcein (FIGS. 12 and 13A-13B), so this dramatic sensitization by p53-reactivation was noteworthy. While SAH-MS1-18 with polymersome delivery was made much more potent, SAH-MS1-18 as a free drug showed no change. This peptide was reportedly highly MCL-1 specific and was reported to cause no non-specific cell membrane disruption. Somehow SAH-MS1-18, without assisted cellular uptake, was unable to exploit the apoptotic priming induced by p53-reactivation. In DLBCL with WTp53, αCD19-PSOM_{SAH-MS1-18} paired with p53-reactivator ATSP-7041 is a potent therapeutic combination in vitro, rivaling cell death sensitivities commonly seen for potent small molecule therapeutics.

### Example 5

### αCD19-PSOM_{calcein} Delivers Calcein to DLBCL In Vivo

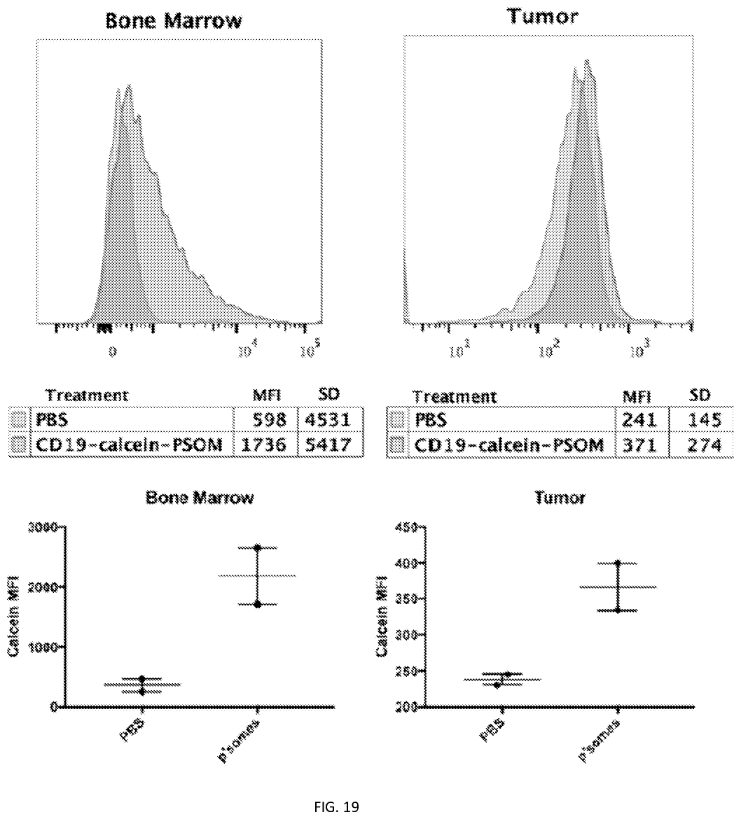

OCI-Ly8 DLBCL cells were engrafted in NSG mice in both a disseminated (i.v.) model and an orthotopic (subcutaneous tumor) model. The mice were treated with one dose of αCD19-PSOMs or vehicle (PBS) six days later, and 24 hours after treatment the mice were sacrificed to analyze the DLBCL cells by flow cytometry. The disseminated OCI-Ly8 cells (CD19+CD20+) were found in the bone marrow but not in the liver and spleen. Both disseminated (bone marrow) and orthotopic (subcutaneous tumor) DLBCL cells had measurable calcein fluorescence compared to vehicle-treated controls (FIG. 19).

### REFERENCES

The following references, some of which are cited above by number, are herein incorporated by reference in their entireties.
[1] Andrei A. Ivanov, Fadlo R. Khuri, and Haian Fu. Targeting protein-protein interactions as an anticancer strategy. Trends in Pharmacological Sciences, 34(7):393-400, July 2013.
[2] Duncan E. Scott, Andrew R. Bayly, Chris Abell, and John Skidmore. Small molecules, big targets: drug discovery faces the protein-protein interaction challenge. Nature Reviews Drug Discovery, 15(8):533-550, August 2016. Number: 8 Publisher: Nature Publishing Group.
[3] James A. Wells and Christopher L. McClendon. Reaching for high-hanging fruit in drug discovery at protein-protein interfaces. Nature, 450(7172):1001-1009, December 2007. Number: 7172 Publisher: Nature Publishing Group.
[4] S. Jones and J. M. Thornton. Principles of protein-protein interactions. Proceedings of the National Academy of Sciences, 93(1):13-20, January 1996. Publisher: National Academy of Sciences Section: Research Article.
[5] Loredana Lo Conte, Cyrus Chothia, and Jöel Janin. The atomic structure of protein-protein recognition sites11 Edited by A. R. Fersht. Journal of Molecular Biology, 285(5):2177-2198, February 1999.
[6] Alan C. Cheng, Ryan G. Coleman, Kathleen T. Smyth, Qing Cao, Patricia Soulard, Daniel R. Caffrey, Anna C. Salzberg, and Enoch S. Huang. Structure-based maximal affinity model predicts small-molecule druggability. Nature Biotechnology, 25(1):71-75, January 2007. Number: 1 Publisher: Nature Publishing Group.
[7] Richard D. Smith, Liegi Hu, Jayson A. Falkner, Mark L. Benson, Jason P. Nerothin, and Heather A. Carlson. Exploring protein-ligand recognition with Binding MOAD. Journal of Molecular Graphics and Modelling, 24(6):414-425, May 2006.
[8] Helen E. Blackwell and Robert H. Grubbs. Highly Efficient Synthesis of Covalently Cross-linked Peptide Helices by Ring Closing Metathesis. Angewandte Chemie International Editions, 37(23):3281-3284, 1998.
[9] Christian E. Schafmeister, Julia Po, and Gregory L. Verdine. An All-Hydrocarbon Cross-Linking System for Enhancing the Helicity and Metabolic Stability of Peptides. Journal of the American Chemical Society, 122(24):5891-5892, June 2000. Publisher: American Chemical Society.
[10] Gregory H. Bird, Federico Bernal, Kenneth Pitter, and Loren D. Walensky. Chapter 22 Synthesis and Biophysical Characterization of Stabilized α-Helices of BCL-2 D mains. In Methods in Enzymology, volume 446 of Programmed Cell Death, The Biology and Therapeutic Implications of Cell Death, Part B, pages 369-386. Academic Press, January 2008.
[11] Gregory L. Verdine and Gerard J. Hilinski. Chapter one—Stapled Peptides for Intracellular Drug Targets. In K. Dane Wittrup and Gregory L. Verdine, editors, Methods in Enzymology, volume 503 of Protein Engineering for Therapeutics, Part B, pages 3-33. Academic Press, January 2012.
[12] Qian Chu, Raymond E. Moellering, Gerard J. Hilinski, Young-Woo Kim, Tom N. Grossmann, Johannes T. H. Yeh, and Gregory L. Verdine. Towards understanding cell penetration by stapled peptides. MedChemComm, 6(1):111-119, 2015. Publisher: Royal Society of Chemistry.
[13] Gregory H. Bird, Emanuele Mazzola, Kwadwo Opoku-Nsiah, Margaret A. Lammert, Marina Godes, Donna S. Neuberg, and Loren D. Walensky. Biophysical determinants for cellular uptake of hydrocarbon-stapled peptide helices. Nature Chemical Biology, 12(10):845-852, October 2016.
[14] Naomi S. Robertson and David R. Spring. Using Peptidomimetics and Constrained Peptides as Valuable Tools for Inhibiting Protein-Protein Interactions. Molecules, 23(4):959, April 2018. Number: 4 Publisher: Multidisciplinary Digital Publishing Institute.
[15] Aileron Therapeutics. A Phase 1/1b Open-Label Study to Determine the Safety and Tolerability of ALRN-6924 Alone and in Combination With Cytarabine (Ara-C) in Patients With Relapsed/Refractory Acute Myeloid Leukemia or Advanced Myelodysplastic Syndrome With Wild-Type TP53. Clinical trial registration NCT02909972, clinicaltrials.gov, November 2019. submitted: Sep. 9, 2016.
[16] Aileron Therapeutics. A Phase 1/2a Open-Label Study to Determine the Safety and Tolerability of ALRN-6924 Alone or in Combination in Patients With Advanced Solid Tumors or Lymphomas Expressing Wild-Type p53 Protein. Clinical trial registration NCT02264613, clinicaltrials.gov, January 2020. submitted: Oct. 7, 2014.
[17] Aileron Therapeutics. A Phase 1b/2 Study of the Dual MDMX/MDM2 Inhibitor, ALRN-6924, for the Prevention of Topotecan-induced Myelosuppression During Treatment for Small Cell Lung Cancer. Clinical trial registration NCT04022876, clinicaltrials.gov, January 2020. submitted: Jul. 12, 2019.
[18] M. D. Anderson Cancer Center. ALRN-6924 and Paclitaxel in Treating Patients With Advanced, Metastatic, or Unresectable Solid Tumors—Full Text View—ClinicalTrials.gov. Technical Report NCT03725436, clinicaltrials.gov, February 2020.
[19] Dana-Farber Cancer Institute. Phase 1 Study of the Dual MDM2/MDMX Inhibitor ALRN-6924 in Pediatric Cancer—Full Text View—ClinicalTrials.gov. Technical Report NCT03654716, clinicaltrials.gov, November 2019.
[20] Clinical Programs. Library Catalog: www.aileronrx.com.
[21] James L. LaBelle, Samuel G. Katz, Gregory H. Bird, Evripidis Gavathiotis, Michelle L. Stewart, Chelsea Lawrence, Jill K. Fisher, Marina Godes, Kenneth Pitter, Andrew L. Kung, and Loren D. Walensky. A stapled BIM peptide overcomes apoptotic resistance in hematologic cancers. The Journal of Clinical Investigation, 122(6):2018-2031, June 2012.
[22] Yong S. Chang, Bradford Graves, Vincent Guerlavais, Christian Tovar, Kathryn Packman, Kwong-Him To, Karen A. Olson, Kamala Kesavan, Pranoti Gangurde, Aditi Mukherjee, Theresa Baker, Krzysztof Darlak, Carl Elkin, Zoran Filipovic, Farooq Z. Qureshi,
Hongliang Cai, Pamela Berry, Eric Feyfant, Xiangguo E. Shi, James Horstick, D. Allen Annis, Anthony M. Manning, Nader Fotouhi, Huw Nash, Lyubomir T. Vassilev, and Tomi K. Sawyer. Stapled α-helical peptide drug development: A potent dual inhibitor of MDM2 and MDMX for p53-dependent cancer therapy. Proceedings of the National Academy of Sciences, 110(36):E3445-E3454, September 2013. Publisher: National Academy of Sciences Section: PNAS Plus.
[23] Raheleh Rezaei Araghi, Gregory H. Bird, Jeremy A. Ryan, Justin M. Jenson, Marina Godes, Jonathan R. Pritz, Robert A. Grant, Anthony Letai, Loren D. Walensky, and Amy E. Keating. Iterative optimization yields Mcl-1-targeting stapled peptides with selective cytotoxicity to Mcl-1-dependent cancer cells. Proceedings of the National Academy of Sciences, 115(5):E886-E895, January 2018.
[24] Yao-Cheng Li, Luo Wei Rodewald, Christian Hoppmann, Ee Tsin Wong, Sylvain Lebreton, Pavel Safar, Marcel Patek, Lei Wang, Kenneth F. Wertman, and Geoffrey M. Wahl. A Versatile Platform to Analyze Low-Affinity and Transient Protein-Protein Interactions in Living Cells in Real Time. Cell Reports, 9(5):1946-1958, December 2014.
[25] Amanda L. Edwards, Evripidis Gavathiotis, James L. LaBelle, Craig R. Braun, Kwadwo A. Opoku-Nsiah, Gregory H. Bird, and Loren D. Walensky. Multimodal Interaction with BCL-2 Family Proteins Underlies the Proapoptotic Activity of PUMA BH3. Chemistry & Biology, 20(7):888-902, July 2013.
[26] Christopher J. Brown, Soo T. Quah, Janice Jong, Amanda M. Goh, Poh C. Chiam, Kian H. Khoo, Meng L. Choong, May A. Lee, Larisa Yurlova, Kourosh Zolghadr, Thomas L. Joseph, Chandra S. Verma, and David P. Lane. Stapled peptides with improved potency and specificity that activate p53. ACS chemical biology, 8(3):506-512, March 2013.
[27] Edward J. Hennessy. Selective inhibitors of Bcl-2 and Bcl-xL: Balancing antitumor activity with on-target toxicity. Bioorganic & Medicinal Chemistry Letters, 26(9):2105-2114, May 2016.
[28] H. Zhang, P. M. Nimmer, S. K. Tahir, J. Chen, R. M. Fryer, K. R. Hahn, L. A. Iciek, S. J. Morgan, M. C. Nasarre, R. Nelson, L. C. Preusser, G. A. Reinhart, M. L. Smith, S. H. Rosenberg, S. W. Elmore, and C. Tse. Bcl-2 family proteins are essential for platelet survival. Cell Death & Differentiation, 14(5):943-951, May 2007. Number: 5 Publisher: Nature Publishing Group.
[29] Kylie D. Mason, Marina R. Carpinelli, Jamie I. Fletcher, Janelle E. Collinge, Adrienne A. Hilton, Sarah Ellis, Priscilla N. Kelly, Paul G. Ekert, Donald Metcalf, Andrew W. Roberts, David C. S. Huang, and Benjamin T. Kile. Programmed Anuclear Cell Death Delimits Platelet Life Span. Cell, 128(6):1173-1186, March 2007.
[30] Wyndham H Wilson, Owen A O'Connor, Myron S Czuczman, Ann S LaCasce, John F Gerecitano, John P Leonard, Anil Tulpule, Kieron Dunleavy, Hao Xiong, Yi-Lin Chiu, Yue Cui, Todd Busman, Steven W Elmore, Saul H Rosenberg, Andrew P Krivoshik, Sari H Enschede, and Rod A Humerickhouse. Navitoclax, a targeted high-affinity inhibitor of BCL-2, in lymphoid malignancies: a phase 1 dose-escalation study of safety, pharmacokinetics, pharmacodynamics, and antitumour activity. The Lancet Oncology, 11(12):1149-1159, December 2010.
[31] L. Gandhi, D. R. Camidge, M. R. De Oliveira, P. Bonomi, D. Gandara, D. Khaira, C. L. Hann, E. M. McKeegan, E. Litvinovich, P. M. Hemken, C. Dive, S. H. Enschede, C. Nolan, Y. L. Chiu, T. Busman, H. Xiong, A. P. Krivoshik, R. Humerickhouse, G. I. Shapiro, and C. M. Rudin. Phase I study of navitoclax (ABT-263), a novel bcl-2 family inhibitor, in patients with small-cell lung cancer and other solid tumors. Journal of Clinical Oncology, 29(7):909-916, 2011.
[32] A. W. Roberts, J. F. Seymour, J. R. Brown, W. G. Wierda, T. J. Kipps, S. L. Khaw, D. A. Carney, S. Z. He, D. C. S. Huang, H. Xiong, Y. Cui, T. A. Busman, E. M. McKeegan, A. P. Krivoshik, S. H. Enschede, and R. Humerickhouse. Substantial susceptibility of chronic lymphocytic leukemia to BCL2 inhibition: Results of a phase I study of navitoclax in patients with relapsed or refractory disease. Journal of Clinical Oncology, 30(5):488-496, 2012.
[33] Bertrand Coiffier, Catherine Thieblemont, Eric Van Den Neste, Gérard Lepeu, Isabelle Plantier, Sylvie Castaigne, Sophie Lefort, Gérald Marit, Margaret Macro, Catherine Sebban, Karim Belhadj, Dominique Bordessoule, Christophe Fermé, and Hervé Tilly. Long-term outcome of patients in the LNH-98.5 trial, the first randomized study comparing rituximab-CHOP to standard CHOP chemotherapy in DLBCL patients: a study by the Groupe d'Etudes des Lymphomes de l'Adulte. Blood, 116(12):2040-2045, September 2010. Publisher: American Society of Hematology.
[34] Jean-François Larouche, Françoise Berger, Catherine Chassagne-Clément, Martine Ffrench, Evelyne Callet-Bauchu, Catherine Sebban, Hervé Ghesquières, Florence Broussais-Guillaumot, Gilles Salles, and Bertrand Coiffier. Lymphoma Recurrence 5 Years or Later Following Diffuse Large B-Cell Lymphoma: Clinical Characteristics and Outcome. Journal of Clinical Oncology, 28(12):2094-2100, March 2010. Publisher: American Society of Clinical Oncology.
[35] Zijun Y. Xu-Monette, Lin Wu, Carlo Visco, Yu Chuan Tai, Alexander Tzankov, Weimin Liu, Santiago Montes-Moreno, Karen Dybkær, April Chiu, Attilio Orazi, Youli Zu, Govind Bhagat, Kristy L. Richards, Eric D. Hsi, X. Frank Zhao, William W. L. Choi, Xiaoying Zhao, J. Han van Krieken, Qin Huang, Jooryung Huh, Weiyun Ai, Maurilio Ponzoni, Andrés J. M. Ferreri, Fan Zhou, Brad S. Kahl, Jane N. Winter, Wei Xu, Jianyong Li, Ronald S. Go, Yong Li, Miguel A. Pins, Michael B. Møller, Roberto N. Miranda, Lynne V. Abruzzo, L. Jeffrey Medeiros, and Ken H. Young. Mutational profile and prognostic significance of TP53 in diffuse large B-cell lymphoma patients treated with R—CHOP: report from an International DLBCL Rituximab-CHOP Consortium Program Study. Blood, 120(19):3986-3996, November 2012. Publisher: American Society of Hematology.
[36] Stefano Monti, Bjoern Chapuy, Kunihiko Takeyama, Scott J. Rodig, Yansheng Hao, Kelly T. Yeda, Haig Inguilizian, Craig Mermel, Treeve Currie, Ahmet Dogan, Jeffery L. Kutok, Rameen Beroukhim, Donna Neuberg, Thomas M. Habermann, Gad Getz, Andrew L. Kung, Todd R. Golub, and Margaret A. Shipp. Integrative Analysis Reveals an Outcome-Associated and Targetable Pattern of p53 and Cell Cycle Deregulation in Diffuse Large B Cell Lymphoma. Cancer Cell, 22(3):359-372, September 2012.
[37] Jenny Zhang, Vladimir Grubor, Cassandra L. Love, Anjishnu Banerjee, Kristy L. Richards, Piotr A. Mieczkowski, Cherie Dunphy, William Choi, Wing Yan Au, Gopesh Srivastava, Patricia L. Lugar, David A. Rizzieri, Anand S. Lagoo, Leon Bernal-Mizrachi, Karen P. Mann, Christopher Flowers, Kikkeri Naresh, Andrew Evens, Leo I. Gordon, Magdalena Czader, Javed I. Gill, Eric D. Hsi, Qingquan Liu, Alice Fan, Katherine Walsh, Dereje Jima, Lisa L. Smith, Amy J. Johnson, John C. Byrd, Micah A. Luftig, Ting Ni, Jun Zhu, Amy Chadburn, Shawn Levy, David Dunson, and Sandeep S. Dave. Genetic heterogeneity of diffuse large B-cell lymphoma. Proceedings of the National Academy of Sciences, 110(4):1398-1403, January 2013. Publisher: National Academy of Sciences Section: Biological Sciences.
[38] Clare M. Adams, Sean Clark-Garvey, Pierluigi Porcu, and Christine M. Eischen. Targeting the Bcl-2 Family in B Cell Lymphoma. Frontiers in Oncology, 8, January 2019.
[39] S. S. Wenzel, M. Grau, C. Mavis, S. Hailfinger, A. Wolf, H. Madle, G. Deeb, B. Dörken, M. Thome, P. Lenz, S. Dirnhofer, F. J. Hernandez-Ilizaliturri, A. Tzankov, and G. Lenz. MCL1 is deregulated in subgroups of diffuse large B-cell lymphoma. Leukemia, 27(6):1381-1390, June 2013. Number: 6 Publisher: Nature Publishing Group.
[40] Ken Kuramoto, Akira Sakai, Kazushi Shigemasa, Yasuo Takimoto, Hideki Asaoku, TakAko Tsujimoto, Kenji Oda, Akiro Kimura, Toshihiro Uesaka, Hiromitsu Watanabe, and Osamu Katoh. High expression of MCL1 gene related to vascular endothelial growth factor is associated with poor outcome in non-Hodgkin's lymphoma. —British Journal of Haematology, 116(1):158-161, 2002. eprint: https://onlinelibrary.wiley.com/doi/pdf/10.1046/j.1365-2141.2002.03253.x.
[41] Georg Lenz and Louis M. Staudt. Aggressive Lymphomas. New England Journal of Medicine, 362(15):1417-1429, April 2010. Publisher: Massachusetts Medical Society eprint: https://doi.org/10.1056/NEJMra0807082.
[42] Georg Lenz, George W. Wright, N. C. Tolga Emre, Holger Kohlhammer, Sandeep S. Dave, R. Eric Davis, Shannon Carty, Lloyd T. Lam, A. L. Shaffer, Wenming Xiao, John Powell, Andreas Rosenwald, German Ott, Hans Konrad Muller-Hermelink, Randy D. Gascoyne, Joseph M. Connors, Elias Campo, Elaine S. Jaffe, Jan Delabie, Erlend B. Smeland, Lisa M. Rimsza, Richard I. Fisher, Dennis D. Weisenburger, Wing C. Chan, and Louis M. Staudt. Molecular subtypes of diffuse large B-cell lymphoma arise by distinct genetic pathways. Proceedings of the National Academy of Sciences, 105(36):13520-13525, September 2008. Publisher: National Academy of Sciences Section: Biological Sciences.
[43] Rameen Beroukhim, Craig H. Mermel, Dale Porter, Guo Wei, Soumya Raychaudhuri, Jerry Donovan, Jordi Barretina, Jesse S. Boehm, Jennifer Dobson, Mitsuyoshi Urashima, Kevin T. Mc Henry, Reid M. Pinchback, Azra H. Ligon, Yoon-Jae Cho, Leila Haery, Heidi Greulich, Michael Reich, Wendy Winckler, Michael S. Lawrence, Barbara A. Weir, Kumiko E. Tanaka, Derek Y. Chiang, Adam J. Bass, Alice Loo, Carter Hoffman, John Prensner, Ted Liefeld, Qing Gao, Derek Yecies, Sabina Signoretti, Elizabeth Maher, Frederic J. Kaye, Hidefumi Sasaki, Joel E. Tepper, Jonathan A. Fletcher, Josep Tabernero, José Baselga, Ming-Sound Tsao, Francesca Demichelis, Mark A. Rubin, Pasi A. Janne, Mark J. Daly, Carmelo Nucera, Ross L. Levine, Benjamin L. Ebert, Stacey Gabriel, Anil K. Rustgi, Cristina R. Antonescu, Marc Ladanyi, Anthony Letai, Levi A. Garraway, Massimo Loda, David G. Beer, Lawrence D. True, Aikou Okamoto, Scott L. Pomeroy, Samuel Singer, Todd R. Golub, Eric S. Lander, Gad Getz, William R. Sellers, and Matthew Meyerson. The landscape of somatic copy-number alteration across human cancers. Nature, 463(7283):899-905, February 2010. Number: 7283 Publisher: Nature Publishing Group.
[44] Ping Zhou, Norman B. Levy, Haiyi Xie, Liping Qian, Chi-Yu Gregory Lee, Randy D. Gascoyne, and Ruth W. Craig. MCL1 transgenic mice exhibit a high incidence of B-cell lymphoma manifested as a spectrum of histologic subtypes. Blood, 97(12):3902-3909, June 2001. Publisher: American Society of Hematology.
[45] Simona Cerritelli, Diana Velluto, and Jeffrey A. Hubbell. PEG-SS—PPS: Reduction-Sensitive Disulfide Block Copolymer Vesicles for Intracellular Drug Delivery. Biomacromolecules, 8(6):1966-1972, June 2007. Publisher: American Chemical Society.
[46] Gregory H. Bird, W. Christian Crannell, and Loren D. Walensky. Chemical Synthesis of Hydrocarbon-Stapled Peptides for Protein Interaction Research and Therapeutic Targeting. Current Protocols in Chemical Biology, 3(3):99-117, 2011.
[47] Young-Woo Kim, Tom N. Grossmann, and Gregory L. Verdine. Synthesis of all-hydrocarbon stapled α-helical peptides by ring-closing olefin metathesis. Nature Protocols, 6(6):761-771, June 2011. Number: 6 Publisher: Nature Publishing Group.
[48] Sean Allen, Omar Osorio, Yu-Gang Liu, and Evan Scott. Facile assembly and loading of theranostic polymersomes via multi-impingement flash nanoprecipitation. Journal of Controlled Release, 262:91-103, September 2017.
[49] Sean D. Allen, Yu-Gang Liu, Sharan Bobbala, Lei Cai, Peter I. Hecker, Ryan Temel, and Evan A. Scott. Polymersomes scalably fabricated via flash nanoprecipitation are non-toxic in non-human primates and associate with leukocytes in the spleen and kidney following intravenous administration. Nano Research, 11(10):5689-5703, October 2018.
[50] Jing Han, Zhengxi Zhu, Haitao Qian, Adam R. Wohl, Charles J. Beaman, Thomas R. Hoye, and Christopher W. Macosko. A simple confined impingement jets mixer for flash nanoprecipitation. Journal of Pharmaceutical Sciences, 101(10):4018-4023, 2012. eprint: https://onlinelibrary.wiley.com/doi/pdf/10.1002/jps.23259.
[51] Sharan Bobbala, Sean David Allen, and Evan Alexander Scott. Flash nanoprecipitation permits versatile assembly and loading of polymeric bicontinuous cubic nanospheres. Nanoscale, 10(11):5078-5088, 2018. Publisher: Royal Society of Chemistry.
[52] Conlin P. O'Neil, Tomoake Suzuki, Davide Demurtas, Andrija Finka, and Jeffrey A. Hubbell. A Novel Method for the Encapsulation of Biomolecules into Polymersomes via Direct Hydration. Langmuir, 25(16):9025-9029, August 2009. Publisher: American Chemical Society.
[53] James Brandon Dixon, Jeffrey A. Hubbell, Conlin P. O'Neil, Melody Swartz, and Diana Velluto. Block copolymers and uses thereof, March 2016. Library Catalog: Google Patents.
[54] A. Pezzutto, B. Dörken, P. S. Rabinovitch, J. A. Ledbetter, G. Moldenhauer, and E. A. Clark. CD19 monoclonal antibody HD37 inhibits anti-immunoglobulin-induced B cell activation and proliferation. The Journal of Immunology, 138(9):2793-2799, May 1987. Publisher: American Association of Immunologists.
[55] Sergey M. Kipriyanov, Olga A. Kupriyanova, Melvyn Little, and Gerhard Moldenhauer. Rapid detection of recombinant antibody fragments directed against cell-surface antigens by flow cytometry. Journal of Immunological Methods, 196(1):51-62, January 1996.
[56] U. E. Schaible, M. D. Kramer, K. Eichmann, M. Modolell, C. Museteanu, and M. M. Simon. Monoclonal antibodies specific for the outer surface protein A (OspA) of Borrelia burgdorferi prevent Lyme borreliosis in severe combined immunodeficiency (scid) mice. Proceedings of the National Academy of Sciences, 87(10):3768-3772, May 1990. Publisher: National Academy of Sciences Section: Research Article.
[57] Wei Ding, Xiaolin Huang, Xiaohua Yang, John J Dunn, Benjamin J Luft, Shohei Koide, and Catherine L Lawson. Structural identification of a key protective B-cell epitope in lyme disease antigen OspA11 Edited by I. A. Wilson. Journal of Molecular Biology, 302(5):1153-1164, October 2000.
[58] Jagath R. Junutula, Helga Raab, Suzanna Clark, Sunil Bhakta, Douglas D. Leipold, Sylvia Weir, Yvonne Chen, Michelle Simpson, Siao Ping Tsai, Mark S. Dennis, Yanmei Lu, Y. Gloria Meng, Carl Ng, Jihong Yang, Chien C. Lee, Eileen Duenas, Jeffrey Gorrell, Viswanatham Katta, Amy Kim, Kevin McDorman, Kelly Flagella, Rayna Venook, Sarajane Ross, Susan D. Spencer, Wai Lee Wong, Henry B. Lowman, Richard Vandlen, Mark X. Sliwkowski, Richard H. Scheller, Paul Polakis, and William Mallet. Site-specific conjugation of a cytotoxic drug to an antibody improves the therapeutic index. Nature Biotechnology, 26(8):925-932, August 2008. Number: 8 Publisher: Nature Publishing Group.
[59] Hongyan Yuan, Ju Li, Gang Bao, and Sulin Zhang. Variable Nanoparticle-Cell Adhesion Strength Regulates Cellular Uptake. Physical Review Letters, 105(13):138101, September 2010.
[60] Loren D. Walensky, Kenneth Pitter, Joel Morash, Kyoung Joon Oh, Scott Barbuto, Jill Fisher, Eric Smith, Gregory L. Verdine, and Stanley J. Korsmeyer. A Stapled BID BH3 Helix Directly Binds and Activates BAX. Molecular Cell, 24(2):199-210, October 2006.
[61] Abbas Hadji, Greta K. Schmitt, Mathew R. Schnorenberg, Lauren Roach, Connie M. Hickey, Logan B. Leak, Matthew V. Tirrell, and James L. LaBelle. Preferential targeting of MCL-1 by a hydrocarbon-stapled BIM BH3 peptide. Oncotarget, 10(58):6219-6233, October 2019.
[62] C. Ploner, R. Kofler, and A. Villunger. Noxa: at the tip of the balance between life and death. Oncogene, 27(1):S84-S92, December 2008. Number: 1 Publisher: Nature Publishing Group.
[63] Jingshan Tong, Peng Wang, Shuai Tan, Dongshi Chen, Zaneta Nikolovska-Coleska, Fangdong Zou, Jian Yu, and Lin Zhang. Mcl-1 Degradation Is Required for Targeted Therapeutics to Eradicate Colon Cancer Cells. Cancer Research, 77(9):2512-2521, May 2017. Publisher: American Association for Cancer Research Section: Therapeutics, Targets, and Chemical Biology.
[64] Thomas Tiller, Eric Meffre, Sergey Yurasov, Makoto Tsuiji, Michel C. Nussenzweig, and Hedda Wardemann. Efficient generation of monoclonal antibodies from single human B cells by single cell RT-PCR and expression vector cloning. Journal of Immunological Methods, 329(1):112-124, January 2008.

We claim:

1. A polymersome comprising:
a plurality of amphiphilic block co-polymers;
a targeting moiety conjugated to a portion of the plurality of amphiphilic block co-polymers on an exterior surface of the polymersome; and
an encapsulated cargo molecule.

2. The polymersome of claim 1, wherein the targeting moiety comprises an antibody or an antibody fragment.

3. The polymersome of claim 1 or claim 2, wherein the targeting moiety binds a cell surface protein.

4. The polymersome of claim 3, wherein the cell surface protein is CD19.

5. The polymersome of any of claims 1-4, wherein the targeting moiety further comprises a cysteine linker.

6. The polymersome of any of claims 1-5, wherein the targeting moiety is conjugated to less than 1% of the plurality of amphiphilic disulfide block co-polymers.

7. The polymersome of any of claims 1-6, wherein the targeting moiety is conjugated to 0.01-1% of the plurality of amphiphilic disulfide block co-polymers.

8. The polymersome of any of claims 1-7, wherein the encapsulated cargo molecule comprises a therapeutic agent, a marker, or a combination thereof.

9. The polymersome of any of claims 1-8, wherein the encapsulated cargo molecule comprises a therapeutic agent.

10. The polymersome of any of claims 1-9, wherein the encapsulated cargo molecule comprises a stapled peptide.

11. The polymersome of any of claims 1-10, wherein the encapsulated cargo molecule comprises a hydrophobic stapled peptide.

12. The polymersome of any of claims 1-11, wherein the encapsulated cargo molecule comprises a hydrocarbon stapled peptide.

13. The polymersome of any of claims 10-12, wherein the stapled peptide contains polar and/or charged functional groups.

14. The polymersome of any of claims 1-13, wherein the amphiphilic block co-polymers comprise a hydrophilic block comprising poly(ethylene glycol) (PEG)

15. The polymersome of any of claims 1-14, wherein the amphiphilic block co-polymers comprise a hydrophobic block comprising poly(propylene sulfide) (PPS).

16. The polymersome of any of claims 1-14, wherein the amphiphilic block co-polymers comprise a linker between a hydrophobic block and a hydrophilic block, wherein the linker is selected from a disulfide and thioether.

17. The polymersome of any of claims 1-16, wherein the polymersome is capable of releasing the encapsulated cargo molecule inside an endosome.

18. A composition comprising the polymersome of any of claims 1-17 and a carrier.

19. A method of treating a disease or disorder in a subject comprising administration of a therapeutically effective amount of the polymersome of any of claims 1-17 or the composition of claim 18 to the subject in need thereof.

20. The method of claim 19, wherein the disease or disorder is cancer.

21. The method of claim 20, further comprising administration of a chemotherapeutic agent.

22. The use of the polymersome of any of claims 1-17, for making a medicament to treat a disease or disorder.
